SEQUENCE LISTING (3 sequences)

SEQ ID NO: 1 - DNA, 38 bp, Artificial
  GFP-AZI forward primer
ggactcgagctatgaaaggatttattgatgatgcaaac

SEQ ID NO: 2 - DNA, 31 bp, Artificial
  GFP-AZI reverse primer
gcagaattcttaagcttcagcggaaaagctg

SEQ ID NO: 3 - protein, 14 aa, Artificial
  C-terminal peptide
CIQLSQEDNFSTEA

(19) United States
(12) Patent Application Publication
(10) Pub. No.: US 2009/0136489 A1
(43) Pub. Date: May 28, 2009

(54) METHODS FOR THE TREATMENT, DIAGNOSIS, AND PROGNOSIS OF CANCER

(75) Inventors: Bruce R. ZETTER, Wayland, MA (US); Sonia KIM, Glenview, IL (US); Ursula MANGOLD, Boston, MA (US)

Correspondence Address: DAVID S. RESNICK, NIXON PEABODY LLP, 100 SUMMER STREET, BOSTON, MA, 02110-2131, US

(73) Assignee: CHILDREN'S MEDICAL CENTER CORPORATION, Boston, MA (US)

(21) Appl. No.: 12/328,832

(22) Filed: Dec. 5, 2008

Related U.S. Application Data
(63) Continuation of application No. 11/301,592, filed on Dec. 13, 2005, now abandoned.
(60) Provisional application No. 60/635,643, filed on Dec. 13, 2004.

(51) Int. Cl. A61K 39/395; A61K 31/7088; A61P 35/00

(52) U.S. Cl. 424/130.1; 514/44

(57) ABSTRACT

We have discovered that antizyme inhibitor (AZI) is expressed at increased levels in highly proliferating cells. We have also discovered that inhibiting antizyme inhibitor, including inhibiting its expression, reduces the growth of cancer cells. The present invention is directed to the use of inhibitors of antizyme inhibitor for the treatment of cancer, the use of antizyme inhibitor for the diagnosis and prognosis of cancer, and methods for identifying novel cancer treatments.

### CROSS-REFERENCE TO RELATED APPLICATIONS

This application is a continuation application of co-pending U.S. Ser. No. 11/301,592 filed Dec. 13, 2005, which is a U.S. utility application and which claims the benefit under 35 U.S.C. § 119(e) of U.S. Provisional Application No. 60/635,643 filed on Dec. 13, 2004, the entire contents of which are incorporated herein.

### CROSS-REFERENCE TO RELATED APPLICATIONS

The present application claims benefit under 35 U.S.C. §119 from the U.S. provisional application No. 60/635,643, filed Dec. 13, 2004, the content of which is herein incorporated by reference in its entirety.

### GOVERNMENT SUPPORT

This invention was made with Government Support under Contract Number CA37393 awarded by the National Institutes of Health. The Government has certain rights in the invention.

### FIELD OF THE INVENTION

The present invention is directed to the use of inhibitors of antizyme inhibitor for the treatment of cancer, the use of antizyme inhibitor for the diagnosis and prognosis of cancer, and methods for identifying novel cancer treatments.

### BACKGROUND OF THE INVENTION

Cancer remains a major health concern. Despite increased understanding of many aspects of cancer, the methods available for its treatment continue to have limited success. First of all, the number of cancer therapies is limited, and none provides an absolute guarantee of success. Second, there are many types of malignancies, and the success of a particular therapy for treating one type of cancer does not mean that it will be broadly applicable to other types. Third, many cancer treatments are associated with toxic side effects. Most treatments rely on an approach that involves killing off rapidly growing cells; however, these treatments are not specific to cancer cells and can adversely affect any dividing healthy cells. Fourth, assessing molecular changes associated with cancerous cells remains difficult. Given these limitations in the current arsenal of anti-cancer treatments, how can the best therapy for a given patient be designed? The ability to detect a malignancy as early as possible, and assess its severity, is extremely helpful in designing an effective therapeutic approach. Thus, methods for detecting the presence of malignant cells and understanding their disease state are desirable, and will contribute to our ability to tailor cancer treatment to a patient's disease.

For example, prostatic carcinoma is the most prevalent form of cancer in males and the second leading cause of cancer death among older males (Boring, et al., Cancer J. Clinicians, 7-26 (1994)). Clinically, radical prostatectomy offers a patient with locally contained disease an excellent chance for cure. If diagnosed after metastases are established, however, prostate cancer is a fatal disease, for which there is no effective treatment that significantly increases survival. The recent development of the prostate specific antigen (PSA) test has dramatically improved diagnosis, allowing earlier detection of prostate cancer and thus earlier treatment (Catalona, et al., J. Urol., 151, 1283-1290 (1994)). Unfortunately, the PSA test does not predict which tumors may progress to the metastatic stage (Cookson, et al., J. Urology 154, 1070-1073 (1995) and Aspinall, et al., J. Urol., 154, 622-628 (1995)). In addition, up to 75% of men who test positive for serum PSA do not have prostate cancer (Caplan & Kratz, Am. J. Clin. Pathol., 117:S104-108 (2002); and Woolf, Int. J. Technol. Assess Health Care, 17(3):275-304 (2001). Such false positives lead to unnecessary medical procedures, and needless anxiety for a large number of men each year. Thus, there is a need in the art for additional biomarkers which can, alone or in combination with PSA or other biomarkers, increase the specificity and sensitivity of prostate cancer diagnosis. Additionally, the treatment and diagnosis of a variety of cancers would be significantly improved by methods for earlier detection, as well as by methods to assess the severity of an individual's cancer.

Another example of an important cancer is gastric cancer, which has a particularly poor prognosis. Although the occurrence of new cases of gastric cancer has diminished in the recent years, gastric cancer is still one of the most common malignancies. In Finland, approximately 250 to 300 new cases of cancer/one million people/year are registered. In the age group of people above 50, there are an estimated 2350 cases of stomach cancer, which is about 3 per mile of the age group population (Finnish Cancer Registry—The Institute for Statistical and Epidemiological Cancer Research 1993). In addition to Finland, there is a high gastric cancer incidence in Iceland, South America and especially in Japan. The prognosis of gastric cancer is usually poor, as there is no specific treatment. Presently the only possibility of successfully treating gastric cancer is its early detection and total removal surgically. Gastric cancer does not necessarily give any symptoms in its early stages. The late appearance of symptoms naturally delays the patient from seeking treatment. On the other hand, the clinical findings in the early stage of gastric cancer are often non-specific. The primary diagnostic method for gastric cancer is presently gastroscopy and biopsies, cell and aspiration cytology associated therewith. As routine gastroscopes are carried out in order to examine symptoms, such as pain in the upper abdomen or bleeding of the gastrointestinal tract, a symptomatic gastric cancer discovered in this manner is often already far advanced and thus inoperable. Attempts have also been made at improving primary diagnostics with various immunological methods, but no sufficiently specific immunological method has been successfully developed.

Antizymes are proteins which bind to ornithine decarboxylase (ODC). ODC is a key enzyme in polyamine biosynthesis. Polyamines play an essential part in cell growth, cell differentiation and protein biosynthesis. Polyamine biosynthesis and the transport of polyamines are regulated in diverse ways at different levels. ODC also plays an apparent role in tumorigenesis, since tumor cells have an increased ODC activity. Thus, for example, overexpression of ODC leads to neoplastic transformation (Auvinen et al. (1992) Nature, 360, 355-358 and Moshier et al., (1993) Cancer Res., 53, 2618-2622). There has been interest in the regulation of ODC activity in order to identify, in the context of tumorigenesis and metastasis, effective substances (ODC effectors), which influence ODC activity. These ODC effectors are able to have an influence on ODC activity directly or indirectly. See for example U.S. Patent Application Publication No. US2003/0165811.

At the protein level, ODC activity and stability are regulated by antizymes (AZ). Antizymes are proteins which bind to ODC, inhibit the enzymatic activity of ODC and stimulate the proteolytic degradation of ODC (Hayashi et al., (1996) TIBS 21, 27-30). In addition, antizymes also regulate polyamine transport into the cell. In addition, there are references in the literature to antitumor activity of antizymes (Feith et al., (2001) Cancer Res., 61, 6073-6081 and Fong et al., (2003) Cancer Res., 63, 3945-3954). In humans, at present four (non-allelic) members of the antizyme family are known, antizyme 1 (e.g. Acc. No. D87914), antizyme 2 (e.g. Acc. No. AF057297), antizyme 3 (e.g. Acc. No. AF175296), and antizyme 4 (e.g. Acc. No. AF293339). Although first thought to bind only to ODC, antizyme 1 has been shown recently to bind and facilitate the degradation of other small proteins, including Smad1 (Gruendler et al., (2001), J. Biol. Chem., 276(49), 46533-43), Snip1 (Lin et al., (2002) BMC Cell Biol., 3(1):15) and cyclin D1 (Newman et al., (2004) J. Biol. Chem. 279(40):41504-11).

The antizyme inhibitor (AZI) has been described as an antizyme regulatory protein that binds with high affinity to antizyme and is able to release ODC bound in the ODC-AZ complex. One screen for genes which are differentially expressed between gastric cancer and healthy human gastric tissues identified AZI as one of 18 genes that were differentially expressed (Jung et al., (2000), Genomics 69, 281-286). However, it is well established that not every gene which is upregulated in one cancer study is an effective target for an anti-cancer therapeutic. Jung et al. merely identified AZI in a screen, but did not demonstrate that inhibition of AZI would have any effect on the proliferation of cancer cells. In addition, Jung et al. discuss AZI only in the context of ODC activity, and do not suggest that it has any other, separate functions.

Despite the substantial attention that has focused on various cancers in recent years, there still exists a strong need for better methods of diagnosis and prognosis, as well as a need for assays to develop better cancer treatments.

### SUMMARY OF THE INVENTION

We have discovered that antizyme inhibitor (AZI) is expressed at increased levels in prostate cancer cells, including in aggressive variants of prostate cancer. We have also discovered that overexpression of Ras leads to an increased expression of antizyme inhibitor. Inhibiting antizyme inhibitor, including preventing its expression, reduces the growth of different cancer cell lines. In addition, we have identified mutant forms and splice variants of antizyme inhibitor.

Accordingly, the invention provides for methods of treatment, diagnosis, and prognosis of cancer, as well as methods to identify novel cancer treatments. One embodiment provides methods for treating cancer by inhibiting AZI, including inhibiting expression of AZI. Another embodiment provides methods of diagnosing cancer by measuring levels of AZI expression, where an increased level of AZI is indicative of cancer. Yet another embodiment provides methods of prognosis of cancer by measuring AZI levels, where a high level of AZI or its variants is indicative of an aggressive form of cancer and thus a poor prognosis. The invention also provides methods for identifying novel cancer treatments by screening for agents which inhibit AZI activity.

One embodiment of the invention provides a method for treating cancer, comprising administering to a subject in need thereof an effective amount of a pharmaceutical comprising an active agent or compound which inhibits antizyme inhibitor, and a pharmaceutically acceptable carrier or diluent. Preferably, the cancer is prostate cancer, lung carcinoma, breast carcinoma, thyroid carcinoma, brain cancers (cerebellum, medulloblastoma, astrocytoma, ependymoma, glioblastoma), ovarian carcinoma, eye cancer (retinoblastoma), muscle (rhapdosarcoma), lymphoma, stomach cancer, liver cancer, colon cancer, kidney cancer, or skin cancer. In one particularly preferred embodiment, the cancer is prostate cancer. In one alternative embodiment the cancer is gastric cancer.

One preferred embodiment of the invention provides a method for treating cancer by using an agent to inhibit antizyme inhibitor, wherein the agent or compound inhibits the antizyme inhibitor by decreasing its expression or function, including transcription, translation or protein function. In this method, preferred agents include DNAs, RNAs, RNA interfering agents, PNAs, small organic molecules, natural products, proteins, antibodies, peptides, and peptidomimetics. One preferred embodiment uses an RNA interfering agent which is a double-stranded, short interfering RNA (siRNA). Preferably, the siRNA is about 15 to about 28 nucleotides in length; even more preferably, about 19 to about 25 nucleotides in length; yet more preferably, about 21 nucleotides in length. In one preferred embodiment of the invention, the siRNA is double-stranded and comprises a 3′ overhand on each strand. The overhang can comprise about 1 to about 6 nucleotides on each strand. In one embodiment, the siRNA can inhibit antizyme inhibitors by transcriptional silencing. In another embodiment, gene therapy using viral vectors which express shRNAs (small hairpin RNAs) can be used to silence AZI.

Another preferred embodiment of the invention provides a method for treating cancer by using an agent to inhibit antizyme inhibitor, wherein the agent or compound inhibits the activity of the antizyme inhibitor.

AZI protein is localized to the centrosome in mammalian cells and participates in the proper segregation of chromosomes during mitosis. Accordingly, AZI or molecules that affect AZI cellular pathways are therefore potential therapeutic agents in prostate cancer and other malignancies.

The invention also provides a method for screening for a compound or agent which modulates the expression of AZI which is useful for the treatment of cancer, comprising providing a cell comprising a reporter plasmid comprising regulatory elements of the AZI gene, including the promoter and the 3′ untranslated region, functionally connected to a nucleic acid encoding a detectable protein; contacting the cell with a test compound or agent; detecting the level of expression of the reporter gene; comparing the level of reporter expression in the presence of the test compound with the level of reporter expression in a control sample in the absence of the test compound, and determining whether or not the test compound increases or decreases the level of reporter expression, wherein a decrease in the level of reporter expression indicates the compound or agent is an antizyme inhibitor.

Another embodiment of the invention provides an assay for screening for agents capable of inhibiting or silencing AZI, comprising the steps of providing a cell transfected with a vector comprising a nucleic acid sequence encoding AZI or a functional fragment thereof; providing an assay for apoptosis or functional mitosis; contacting the cell with a test agent; detecting the amount of functional mitosis in the sample; and comparing the amount of functional mitosis expression in the presence of the test compound with the level of functional mitosis in a control sample in the absence of the test compound; and determining whether or not the test compound increases or decreases the level of functional mitosis, wherein a decrease in the level of functional mitosis indicates the compound or agent is an inhibitor of antizyme inhibitor.

The present invention also provides a method of diagnosing a disease, such as cancer, for example, prostate cancer in a patient, comprising obtaining a test sample from a patient; measuring the level of antizyme inhibitor and/or its variants in the test sample; and comparing the level of antizyme inhibitor in the test sample with the level of antizyme inhibitor present in a normal control sample; wherein a higher level of antizyme inhibitor in the test sample as compared to the level in the normal control sample is indicative of cancer. The test sample and said normal control sample can be samples of blood, tissue, serum, stool, urine, sputum, cerebrospinal fluid, or supernatant from cell lysate.

In one preferred embodiment, the level of antizyme inhibitor is measured by measuring the levels of antizyme inhibitor mRNA. Preferably, the mRNA is detected by the use of an RNA dependent polymerase chain reaction, including but not limited to quantitative RT-PCR. In another preferred embodiment, the mRNA is detected by Northern blot hybridization analysis by hybridizing mRNA from a test sample or a control sample to an antizyme inhibitor nucleotide probe. In other preferred embodiment, the level of mRNA is detected by nucleic acid microarray analysis.

In another preferred embodiment, the level of antizyme inhibitor can be measured by measuring the levels of antizyme inhibitor protein. Preferred methods to measure protein levels include quantitative immunoblot techniques and ELISA.

Another preferred method for measuring the level of antizyme is to use an antibody or antibody fragment which selectively binds antizyme inhibitor. Preferably, the antibody or antibody fragment is detectably labeled.

The present invention also provides methods for prognostic evaluation of a patient suspected of having or having cancer comprising measuring the level of antizyme inhibitor and/or its variants in a test sample obtained from a patient; comparing the level in the test sample to a range of antizyme inhibitor known to be present in a biological sample obtained from a normal patient who does not have cancer; and evaluating the prognosis of said patient based on the comparison, wherein a high level of antizyme inhibitor in the test sample indicates an aggressive form of cancer and therefore a poor prognosis.

The present invention also provides a kit for measuring antizyme inhibitor levels and/or its variants, comprising separate vials containing antibodies, or antibody fragments, which selectively bind human antizyme inhibitor.

### BRIEF DESCRIPTION OF THE DRAWINGS

FIG. 1 shows that siRNA against AZI effects growth and cell shape of AT2.1 cells. AT2.1 cells, which are a prostate cell carcinoma cell line, were transiently transfected with siRNA against AZI or a scrambled control (scramble II Duplex, Dharmacon). Phase pictures were taken two days after transfection. Cells were quantified using a Coulter cell counter.

FIG. 2 is a schematic that summarizes the interation of ODC, AZ, and AZI. ODC, the rate limiting enzyme of polyamine biosynthesis, is active as a homodimer. ODC is negatively regulated through antizyme (AZ, which binds the ODC monomer and promotes degradation of ODC through the 26S proteasome. In addition, AZ also inhibits polyamine uptake. AZ targets proteins for degradation without ubiquitination. There are two different substrate targeting pathways for the 26S proteosome: an ubiquitin-dependent pathway for poly-ubiquitinated proteins, and an antizyme-dependent pathway for ODC (Murakami et al., (1992), Nature, 360(6404):597-9). AZ is in turn negatively regulated by the antizyme inhibitor (AZI), which binds AZ with higher affinity than ODC. AZI lacks PEST domains and is not targeted to the 26S proteasome by AZ.

FIG. 3 shows that the effect of AZI overexpression on proliferation is not entirely dependent on the activation of ODC. AT2.1 cells were transfected with either a control vector, pTEH (diamonds), the vector carrying the gene encoding wildtype AZI (AZIwt, shown as squares), or an AZI mutant that does not bind AZ (AZImut, shown as triangles), due to a deletion of the region encoding amino acids 114-140. Stable transfectants were selected and cells were grown for five days, with growth measures at each day. The overexpression of AZI in AT2.1 cells was associated with an increased rate of proliferation compared to the control cells; this effect on proliferation was not dependent on the ability of AZI to bind AZ.

FIGS. 4A-4R show that AZI localizes to centrosomes throughout the cell division cycle. Immunofluorescence analysis of non synchronized NIH-3T3 cells stained with antibodies against AZI (mouse monoclonal) and pericentrin (rabbit polyclonal). Nuclei were stained with DAPI. During telophase, an AZI signal also appeared adjacent to the intercellular bridge (white arrow).

FIGS. 5A-5O show that AZI localizes to centrosomes in different mammalian cell lines. Immunofluorescence analysis of AZI in human primary cells (HUVEC, HFF) or U2OS (human), AT2.1 (rat) and NIH-3T3 (mouse) cell lines. Centrosomes were visualized using a rabbit polyclonal antibody against pericentrin. AZI was visualized using the mouse monoclonal antibody. Nuclei were stained with DAPI.

FIG. 6 shows that silencing of AZI leads to a decrease in centrosome abnormalities. U2OS and AT2.1 cells were transiently transfected with siRNA against AZI (AZI-139 and AZI-pool) and harvested after 48 h. AZI139 specifically targets an N-terminal sequence of the rat AZI coding sequence. The commercially available AZIpool (Smartpool) consists out of four pooled siRNAs directed against human AZI. Immunoblot analysis shows that AZI protein expression is greatly reduced in siRNA-treated cells. A non-specific scrambled siRNA was used as control (SCR).

FIG. 7A shows immunofluorescence analysis of AZI levels in U2OS cells treated with siRNA against AZI or control siRNA (SCR). Cells were stained for AZI (mouse monoclonal) and pericentrin (rabbit polyclonal). The upper panel shows centrosomal AZI signal in a cell treated with scrambled siRNA. The lower panel shows a cell which was treated with siRNA against AZI and has undetectable AZI expression at the centrosome (arrow). The level of AZI knockdown was similar to the results obtained by immunoblot analysis. FIGS. 7B and 7C show quantitative analysis of centrosome abnormalities in U2OS and AT2.1 cells treated with siRNA against AZI (AZI-pool, AZI139) or a scrambled control siRNA. Cells were plated on coverslips, treated with the corresponding siRNAs the following day and fixed 48 h later. Centrosomes were visualized by immunofluorescence staining for γ-tubulin. Bars represent the mean of two independent experiments±s.d. At least 100-150 cells were evaluated per experiment.

FIG. 8 shows that silencing of antizyme leads centrosome amplification. NIH-3T3 and AT2.1 cells were transiently transfected with siRNA against antizyme 1 (AZ-1 and AZ-2) and harvested 48 h later. AZ-1 specifically targets an N-terminal sequence of the rat/mouse antizyme 1 and AZ-2 targets a C-terminal sequence of the rat/mouse antizyme 1. Immunoblot analysis shows that antizyme 1 protein expression is greatly reduced in siRNA-treated cells. A non-specific scrambled siRNA was used as control (SCR)

FIG. 9 shows quantitation of increase in the number of cells with abnormal centrosome number. Quantitative analysis of centrosome abnormalities in NIH-3T3 and AT2.1 cells treated with siRNA against antizyme 1 (AZ-1, AZ-2) or control scrambled siRNA. Cells were plated onto coverslips, treated with the corresponding siRNAs the following day and fixed 48 h later. Centrosomes were stained for γ-tubulin and visualized by immunofluorescence Bars represent the mean of two independent experiments±s.d. At least more then 150 cells were evaluated per experiment.

FIG. 10 shows stable overexpression of AZI leads to centrosome amplification in U2OS cells. Immunoblot analysis of GFP-AZI overexpressing U2OS cells. Three independent GFP-AZI clones (C1-3) were analysed using antibodies against GFP, AZI (mouse monoclonal), cyclin D1, ODC, antizyme 1 and aurora A. Actin was used as loading control. The negative control represents lysate from parental U2OS cells.

FIG. 11 shows a TUNEL assay of cells treated for 48 hours with AZI-RNAi or a scrambled control RNA. NIH-3T3 cells were used.

FIG. 12 shows parental U2OS cells and two AZI-overexpressing clones that were analysed for aurora A kinase activity. Lysates of non-synchronized cells were subjected to a non-radioactive immunoassay for aurora A kinase activity using an anti-phospho-Lats2 serine-83 monoclonal antibody and peroxidase-coupled anti-mouse antibody as a reporter molecule. Assay was repeated with all samples assayed in duplicates.

FIG. 13 shows AZI overexpressing cells (clone 3) and parental U2OS cells that were grown in 0.5% FBS containing media and were treated for 24 hours with either 0.01 mM or 0.05 mM DFMO Polyamine levels were measured as described in Materials and Methods. Mean values from duplicate samples±s.d. are shown.

FIGS. 14A and 14B show that overexpression of antizyme 1 leads to a decrease in centrosome abnormalities. FIG. 14A shows that immunoblot analysis of GFP-antizyme 1 overexpressing U2OS cells. Two independent GFP-antizyme 1 clones were analysed using antibodies against GFP or antizyme 1 (mouse monoclonal). Actin was used as loading control. The negative control represents lysate from parental U2OS cells. FIG. 14B shows quantitative analysis of centrosomal abnormalities in U2OS cells stably overexpressing GFP-antizyme 1. Two independent antizyme 1 overexpressing clones were analysed for centrosome abnormalities. The combined result of three independent stable GFP clones was used as control. Centrosomes were stained for γ-tubulin and visualized by immunofluorescence. Bars represent the means of three independent experiments±s.d.

FIG. 15 shows quantitative analysis of centrosome abnormalities in U2OS cells upon treatment with hydroxyurea. U2OS wild-type and U2OS-antizyme (U2OS-AZ) overexpressing cells were transfected with siRNA against AZI (Smartpool) or scrambled control siRNA. 24 h after transfection cells were treated with 2 mM hydroxyurea for an additional 48 h. Centrosomes were visualized by immunofluorescence staining for γ-tubulin. Bars represent the mean of two independent experiments±s.d. At least 150-200 cells were evaluated for each experiment.

### DETAILED DESCRIPTION OF THE INVENTION

We have discovered that antizyme inhibitor (AZI) is expressed at increased levels in highly proliferating cells, including prostate cancer cell lines. We have also discovered that AZI regulates cell growth. Silencing AZI leads to reduced cell proliferation and apoptosis, and increased numbers of centrosomes, which is associated with polyploidy and many types of cancer. We have also discovered that the AZI protein localizes to the centrosome within the cell.

These results indicate that disruption of the function or expression of AZI is an effective target for treatment of cancers which overexpress AZI. Accordingly, the present invention provides a method of treating neoplastic cells expressing human AZI by administering to the cell an effective amount of a compound or agent that suppresses the production or activity of the human AZI. Preferably, the compound interferes with the expression of the human AZI gene or protein. Such compounds include, for example, siRNAs, antisense oligonucleotides, ribozymes, RNAi, and antibodies.

These results also indicate that increased expression of AZI and its variants has a correlation to disease state in cancers, including prostate cancer. Accordingly, assaying for enhanced levels of transcript or gene product can be used in both in a diagnostic manner as well as in a prognostic manner for particular cancers which overexpress AZI. The present invention provides a method of diagnosing cancer in a patient, such as prostate cancer. The method comprises determining or measuring the levels of AZI in a biological test sample obtained from the patient and comparing the observed level of AZI with the level of AZI in a normal control sample of the same type. Said determination or measurement can be performed from altered AZI levels of the patient sample. Higher levels of AZI in the test sample, as compared to the AZI levels in a normal control sample, is indicative of cancer. Additionally, AZI can be used alone or in conjunction with other cancer markers, e.g. prostate specific antigen (PSA) and thymosin β15, in the diagnosis and prognosis of cancer. For example, PSA is a widely used diagnostic for prostate cancer, however detection of PSA leads to many false positives as well as false negatives. Monitoring the presence of AZI along with levels of PSA can be used to increase the specificity and sensitivity of prognosis of prostate cancer. In addition, AZI can be used in the diagnosis and prognosis of other cancers as well that express increased levels of AZI, including in conjunction with other cancer markers.

Another embodiment of the present invention provides methods of prognosis of cancer by determining AZI levels, where a high level of AZI or its variants is indicative of an aggressive form of cancer and thus a poor prognosis. The invention also provides methods for identifying novel cancer treatments by screening for agents which inhibit AZI.

Determination of AZI levels can be performed from AZI level measurement. There measurements can be done by the diagnostic facility or out sourced to other facilities in the U.S. or other countries.

As used herein, the term “aggressive” or “invasive” with respect to cancer refers to the proclivity of a tumor for expanding beyond its boundaries into adjacent tissue (Darnell, J. (1990), Molecular Cell Biology, Third Ed., W.H. Freeman, NY). Invasive cancer can be contrasted with organ-confined cancer wherein the tumor is confined to a particular organ. The invasive property of a tumor is often accompanied by the elaboration of proteolytic enzymes, such as collagenases, that degrade matrix material and basement membrane material to enable the tumor to expand beyond the confines of the capsule, and beyond confines of the particular tissue in which that tumor is located.

The term “metastasis”, as used herein, refers to the condition of spread of cancer from the organ of origin to additional distal sites in the patient. The process of tumor metastasis is a multistage event involving local invasion and destruction of intercellular matrix, intravasation into blood vessels, lymphatics or other channels of transport, survival in the circulation, extravasation out of the vessels in the secondary site and growth in the new location (Fidler, et al., Adv. Cancer Res. 28, 149-250 (1978), Liotta, et al., Cancer Treatment Res. 40, 223-238 (1988), Nicolson, Biochim. Biophy. Acta 948, 175-224 (1988) and Zetter, N. Eng. J. Med. 322, 605-612 (1990)). Increased malignant cell motility has been associated with enhanced metastatic potential in animal as well as human tumors (Hosaka, et al., Gann 69, 273-276 (1978) and Haemmerlin, et al., Int. J. Cancer 27, 603-610 (1981)).

As used herein, a “biological sample” refers to a sample of biological material obtained from a patient, preferably a human patient, including a tissue, a tissue sample, a cell sample (e.g., a tissue biopsy, such as, an aspiration biopsy, a brush biopsy, a surface biopsy, a needle biopsy, a punch biopsy, an excision biopsy, an open biopsy, an incision biopsy or an endoscopic biopsy), and a tumor sample. Biological samples can also be biological fluid or excruent samples e.g., blood, urine, or stool, sputum or saliva.

The present invention also encompasses the use of isolates of a biological sample in the methods of the invention. As used herein, an “isolate” of a biological sample (e.g., an isolate of a tissue or tumor sample) refers to a material or composition (e.g., a biological material or composition) which has been separated, derived, extracted, purified or isolated from the sample and preferably is substantially free of undesirable compositions and/or impurities or contaminants associated with the biological sample.

As used herein, a “tissue sample” refers to a portion, piece, part, segment, or fraction of a tissue which is obtained or removed from an intact tissue of a subject, preferably a human subject.

As used herein, a “tumor sample” refers to a portion, piece, part, segment, or fraction of a tumor, for example, a tumor which is obtained or removed from a subject (e.g., removed or extracted from a tissue of a subject), preferably a human subject.

As used herein, a “primary tumor” is a tumor appearing at a first site within the subject and can be distinguished from a “metastatic tumor” which appears in the body of the subject at a remote site from the primary tumor.

### Cancer Patients/Subjects for Administration

The presence of AZI is indicative of aggressive cancer. Accordingly, the present invention provides methods for the treatment of cancer comprising to a patient in need thereof an agent or compound that inhibits AZI.

One embodiment of the invention provides a method for treating cancer, comprising administering to a subject in need thereof an effective amount of a pharmaceutical comprising an active agent or compound which inhibits antizyme inhibitor, and a pharmaceutically acceptable carrier or diluent.

In one preferred embodiment, the pharmaceutical compositions and therapeutic methods of the present invention can be used to treat any patient with a cancer which expresses a higher level of AZI than the amount of AZI present in a normal control sample. A “high level” of AZI refers to amounts of AZI that are at least about 2 to 3 fold greater than the amounts of AZI present in normal control samples, preferably about 5 fold to about 6 fold or greater. The cancer is prostate cancer. Alternatively the cancer is gastric cancer.

It has also been found that antizyme is differentially expressed between healthy and tumor tissues; thus, these cancers represent additional target populations for the methods of the present invention. Such preferred cancers include breast cancer (see US Patent Application No. 20040002067), prostate cancer (see US Patent Application No. 20030215835), bladder cancer (see US Patent Application Nos. 20040038207 and 20040076955), and ovarian cancer (see US Patent Application No. 20040005563 and Yanaihara et al., Int. J. Oncol. 23:567-75 (2003)).

In the methods of the present invention, one first screens the patient to determine the presence and type of a cancer that expresses high levels of AZI, as described in detail below. Preferably, one also screens the patient to determine what stage of cancer the patient has.

Within such methods, the pharmaceutical compositions described herein are administered to a host. Preferably, the host is a mammal. Preferred mammals include primates such as humans and chimpanzees, domestic animals such as horses, cows, pigs, etc. and pets such as dogs and cats. More preferably, the host mammal is a primate or domestic animal. Still more preferably the host mammal is a human.

Pharmaceutical compositions of the present invention may be administered either prior to or following surgical removal of primary tumors and/or treatment such as administration of radiotherapy or conventional chemotherapeutic drugs. As discussed below, administration of the pharmaceutical compositions may be by any suitable method, including administration by intravenous, intraperitoneal, intramuscular, subcutaneous, intranasal, intradermal, anal, vaginal, topical and oral routes.

Agents or compounds which inhibit AZI which can be used in the pharmaceutical compositions of the present invention include agents which downregulate AZI by decreasing its transcription, leading to decreased gene expression and decreased levels of AZI protein, as well as agents which inhibit the activity of AZI.

### Downregulation of AZI

AZI is therapeutically useful to treat prostate cancer because we have discovered that AZI is overexpressed in highly proliferating cancer cells and that downregulation of AZI inhibits growth of cells.

The present invention provides a method for treating cancer by using an agent to inhibit antizyme inhibitor, wherein the agent or compound downregulates AZI. In one preferred embodiment, the agent inhibits AZI by decreasing expression or function of protein. In this method, preferred agents include DNAs, RNAs, RNA interfering agents, PNAs, small organic molecules, natural products, proteins, antibodies, peptides, and peptidomimetics.

Preferably, AZI expression is inhibited in vivo by the use of any method which results in decreased expression of the gene encoding AZI, including but not limited to RNAi technology. RNA interference or “RNAi” is a term initially coined by Fire and co-workers to describe the observation that double-stranded RNA (dsRNA) can block gene expression when it is introduced into worms (Fire et al. (1998) Nature 391, 806-811). “RNA interference (RNAi)” is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target genes. See for example U.S. Patent Application Nos: 20030153519A1; 20030167490A1; and U.S. Pat. Nos. 6,506,559; 6,573,099, which are herein incorporated by reference in their entirety.

Isolated RNA molecules specific to antizyme inhibitor mRNA, which mediate RNAi, are antagonists useful in the method of the present invention. In one embodiment, the RNA interfering agents used in the methods of the invention, e.g., the siRNAs used in the methods of the invention, have been shown to be taken up actively by cells in vivo following intravenous injection, e.g., hydrodynamic injection, without the use of a vector, illustrating efficient in vivo delivery of the RNA interfering agents, e.g., the siRNAs used in the methods of the invention. In one embodiment, siRNAs are human AZI sequences corresponding to the rat/mouse AZI sequence 139-159 or AZI SMART POOL mixture (Dharmacon) of such siRNAs.

Other strategies for delivery of the RNA interfering agents, e.g., the siRNAs or shRNAs of used in the methods of the invention, may also be employed, such as, for example, delivery by a vector, e.g., a plasmid or viral vector, e.g., a lentiviral vector. Other delivery methods include delivery of the RNA interfering agents, e.g., the siRNAs or shRNAs of the invention, using a basic peptide by conjugating or mixing the RNA interfering agent with a basic peptide, e.g., a fragment of a TAT peptide, mixing with cationic lipids or formulating into particles.

The RNA interfering agents, e.g., the siRNAs of the invention, can be introduced directly into the subject in such a manner that they are directed to and taken up by the target cells and regulate or promote RNA interference of AZI. The RNA interfering agents, e.g., the siRNAs of the invention, may be delivered singly, or in combination with other RNA interfering agents, e.g., siRNAs, such as, for example siRNAs directed to other cellular genes, e.g., apoptosis-related genes. The RNA interfering agents, e.g., siRNAs of the invention may also be administered in combination with other pharmaceutical agents which are used to treat or prevent cancer.

An “RNA interfering agent” as used herein, is defined as any agent which interferes with or inhibits expression of a target gene or genomic sequence by RNA interference (RNAi). Such RNA interfering agents include, but are not limited to, nucleic acid molecules including RNA molecules which are homologous to the target gene or genomic sequence, or a fragment thereof, short interfering RNA (siRNA), short hairpin or small hairpin RNA (shRNA), and small molecules which interfere with or inhibit expression of a target gene by RNA interference (RNAi). The target gene of the present invention is the gene encoding AZI.

As used herein, “inhibition of target gene expression” includes any decrease in expression or protein activity or level of the target gene or protein encoded by the target gene. The decrease may be of at least 30%, 40%, 50%, 60%, 70%, 80%, 90%, 95% or 99% or more as compared to the expression of a target gene or the activity or level of the protein encoded by a target gene which has not been targeted by an RNA interfering agent.

“Short interfering RNA” (siRNA), also referred to herein as “small interfering RNA” is defined as an agent which functions to inhibit expression of a target gene, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 29 nucleotides, more preferably about 19 to about 25 nucleotides in length, and more preferably about 19, 20, 21, or 22 nucleotides in length, alternatively the length of the siRNA can be 27-29 nucleotides long. The dsRNA may also contain a 3′ and/or 5′ overhang on each strand having a length of about 0, 1, 2, 3, 4, 5, or 6 nucleotides. The length of the overhang is independent between the two strands, i.e., the length of the overhang on one strand is not dependent on the length of the overhang on the second strand. In one embodiment, the siRNA can inhibit antizyme inhibitors by transcriptional silencing. Preferably the siRNA is capable of promoting RNA interference through degradation or specific post-transcriptional gene silencing (PTGS) of the target messenger RNA (mRNA).

siRNAs also include small hairpin (also called stem loop) RNAs (shRNAs). In one embodiment, these shRNAs are composed of a short (e.g., about 19 to about 25 nucleotide) antisense strand, followed by a nucleotide loop of about 5 to about 9 nucleotides, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501, incorporated be reference herein).

An siRNA may be substantially homologous to the target AZI gene or genomic sequence, or a fragment thereof. As used herein, the term “homologous” is defined as being substantially identical, sufficiently complementary, or similar to the target mRNA, or a fragment thereof, to effect RNA interference of the target. In addition to native RNA molecules, RNAs suitable for inhibiting or interfering with the expression of a target sequence include RNA derivatives and analogs. siRNA molecules need not be limited to those molecules containing only RNA, but, for example, further encompasses chemically modified nucleotides and non-nucleotides, and also include molecules wherein a ribose sugar molecule is substituted for another sugar molecule or a molecule which performs a similar function. Moreover, a non-natural linkage between nucleotide residues may be used, such as a phosphorothioate linkage. The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatives with a variety of groups.

Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases may also be modified. Any modified base useful for inhibiting or interfering with the expression of a target sequence may be used. For example, halogenated bases, such as 5-bromouracil and 5-iodouracil can be incorporated. The bases may also be alkylated, for example, 7-methylguanosine can be incorporated in place of a guanosine residue. Non-natural bases that yield successful inhibition can also be incorporated. In a preferred embodiment, the RNA is stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly enhances the nuclease resistance of the overhang in tissue culture medium.

Antizyme inhibitor expression may also be inhibited in vivo by the use of any method which results in decreased transcription of the gene encoding AZI. One embodiment uses antisense technology. Gene expression can be controlled through triple-helix formation or antisense DNA or RNA, both of which methods are based on binding of a polynucleotide to DNA or RNA. An antisense nucleic acid molecule which is complementary to a nucleic acid molecule encoding AZI can be designed based upon the isolated nucleic acid molecules encoding AZI by means known to those in the art.

Design and Preparation of siRNA Molecules

Synthetic siRNA molecules, including shRNA molecules, of the present invention can be obtained using a number of techniques known to those of skill in the art. One preferred siRNA is described in detail below in Example 1. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al. (2001) Nature 411:494-498; Elbashir, S. M., W. Lendeckel and T. Tuschl (2001) Genes & Development 15:188-200; Harborth, J. et al. (2001) J. Cell Science 114:4557-4565; Masters, J. R. et al. (2001) Proc. Natl. Acad. Sci., USA 98:8012-8017; and Tuschl, T. et al. (1999) Genes & Development 13:3191-3197). Alternatively, several commercial RNA synthesis suppliers are available including, but not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, siRNA molecules are not overly difficult to synthesize and are readily provided in a quality suitable for RNAi. In addition, dsRNAs can be expressed as stem loop structures encoded by plasmid vectors, retroviruses and lentiviruses (Paddison, P. J. et al. (2002) Genes Dev. 16:948-958; McManus, M. T. et al. (2002) RNA 8:842-850; Paul, C. P. et al. (2002) Nat. Biotechnol. 20:505-508; Miyagishi, M. et al. (2002) Nat. Biotechnol. 20:497-500; Sui, G. et al. (2002) Proc. Natl. Acad. Sci., USA 99:5515-5520; Brummelkamp, T. et al. (2002) Cancer Cell 2:243; Lee, N. S., et al. (2002) Nat. Biotechnol. 20:500-505; Yu, J. Y., et al. (2002) Proc. Natl. Acad. Sci., USA 99:6047-6052; Zeng, Y., et al. (2002) Mol. Cell 9:1327-1333; Rubinson, D. A., et al. (2003) Nat. Genet. 33:401-406; Stewart, S. A., et al. (2003) RNA 9:493-501). These vectors generally have a polIII promoter upstream of the dsRNA and can express sense and antisense RNA strands separately and/or as a hairpin structures. Within cells, Dicer processes the short hairpin RNA (shRNA) into effective siRNA.

The targeted region of the siRNA molecule of the present invention can be selected from a given target AZI gene sequence, beginning from about 25 to 50 nucleotides, from about 50 to 75 nucleotides, or from about 75 to 100 nucleotides downstream of the start codon. Nucleotide sequences may contain 5′ or 3′ UTRs and regions nearby the start codon. One method of designing a siRNA molecule of the present invention involves identifying the 23 nucleotide sequence motif AA(N19)TT (where N can be any nucleotide) and selecting hits with at least 25%, 30%, 35%, 40%, 45%, 50%, 55%, 60%, 65%, 70% or 75% G/C content. The “TT” portion of the sequence is optional. Alternatively, if no such sequence is found, the search may be extended using the motif NA(N21), where N can be any nucleotide. In this situation, the 3′ end of the sense siRNA may be converted to TT to allow for the generation of a symmetric duplex with respect to the sequence composition of the sense and antisense 3′ overhangs. The antisense siRNA molecule may then be synthesized as the complement to nucleotide positions 1 to 21 of the 23 nucleotide sequence motif. The use of symmetric 3′ TT overhangs may be advantageous to ensure that the small interfering ribonucleoprotein particles (siRNPs) are formed with approximately equal ratios of sense and antisense target RNA-cleaving siRNPs (Elbashir et al. (2001) supra and Elbashir et al. 2001 supra). Analysis of sequence databases, including but not limited to the NCBI, BLAST, Derwent and GenSeq as well as commercially available oligosynthesis companies such as OLIGOENGINE®, may also be used to select siRNA sequences against EST libraries to ensure that only one gene is targeted.

### Delivery of RNA Interfering Agents

Methods of delivering RNA interfering agents, e.g., an siRNA of the present invention, or vectors containing an RNA interfering agent, to the target cells, e.g., tumor cells, for uptake include injection of a composition containing the RNA interfering agent, e.g., an siRNA, or directly contacting the cell, e.g., a tumor cell, or tissue, with a composition comprising an RNA interfering agent, e.g., an siRNA. In another embodiment, RNA interfering agents, e.g., an siRNA may be injected directly into any vein or artery, via, e.g., hydrodynamic injection. Administration may be by a single injection or by two or more injections. One can also use liposonnal mixtures of siRNAs.

A viral-mediated delivery mechanism may also be employed to deliver siRNAs to cells in vitro and in vivo as described in Xia, H. et al. (2002) Nat Biotechnol 20(10):1006). Plasmid- or viral-mediated delivery mechanisms of shRNA may also be employed to deliver shRNAs to cells in vitro and in vivo as described in Rubinson, D. A., et al. ((2003) Nat. Genet. 33:401-406) and Stewart, S. A., et al. ((2003) RNA 9:493-501). Other methods of introducing siRNA molecules of the present invention to target cells, e.g., tumor cells, include a variety of art-recognized techniques including, but not limited to, calcium phosphate or calcium chloride co-precipitation, DEAE-dextran-mediated transfection, lipofection, or electroporation as well as a number of commercially available transfection kits (e.g., OLIGOFECTAMINE® Reagent from Invitrogen) (see, e.g. Sui, G. et al. (2002) Proc. Natl. Acad. Sci. USA 99:5515-5520; Calegari, F. et al. (2002) Proc. Natl. Acad. Sci., USA Oct. 21, 2002 [electronic publication ahead of print]; J-M Jacque, K. Triques and M. Stevenson (2002) Nature 418:435-437; and Elbashir, S. M et al. (2001) supra). Suitable methods for transfecting a target tumor cell can also be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals. The efficiency of transfection may depend on a number of factors, including the cell type, the passage number, the confluency of the cells as well as the time and the manner of formation of siRNA- or shRNA-liposome complexes (e.g., inversion versus vortexing). These factors can be assessed and adjusted without undue experimentation by one with ordinary skill in the art.

The RNA interfering agents, e.g., the siRNAs or shRNAs of the invention, may be introduced along with components that perform one or more of the following activities: enhance uptake of the RNA interfering agents, e.g., siRNA, by the tumor cell inhibit annealing of single strands, stabilize single strands, or otherwise facilitate delivery to the target cell and increase inhibition of the target gene, antizyme inhibitor.

The RNA interfering agents, e.g., siRNA, may be directly introduced into the cell, or introduced extracellularly into a cavity, interstitial space, into the circulation of an organism, introduced orally, or may be introduced by bathing a cell or organism in a solution containing the RNA interfering agent, e.g., an siRNA. RNA interfering agents, e.g., an siRNA, may also be introduced into cells via topical application to a mucosal membrane or dermally. Vascular or extravascular circulation, the blood or lymph system, and the cerebrospinal fluid are also sites where the agents may be introduced.

### Inhibition of AZI Activity

Another preferred embodiment of the invention provides a method for treating cancer by using an agent to inhibit antizyme inhibitor, wherein the agent or compound inhibits the activity of the antizyme inhibitor. One can treat a range of afflictions or diseases associated with expression of the protein by directly blocking the activity of the protein. This can be accomplished by a range of different approaches, including the use of antibodies, small molecules, and antagonists. One preferred method of inhibiting AZI provides an antizyme (AZ) peptide which would competitively bind and thus inhibit AZI.

Means for measuring antizyme inhibitor activity are well known to those skilled in the art and any such method can be used. For example, one method for determining AZI activity is described in pending published application, number US2003/0165811, in which cells which express ornithine decarboxylase (ODC), antizyme and antizyme inhibitor are cultivated on a polyamine-free medium in the presence and absence of putrescine, and the growth of said cells is determined. AZI activity can also be measured indirectly by measuring ODC, whose activity can be measured by determining the release of ¹⁴CO₂ from radioactively labeled ornithine.

### Diagnosis and Prognosis

The present invention also provides methods for diagnosis of cancer in a patient. The methods involve measuring levels of AZI in a test sample obtained from a patient, suspected of having cancer, and comparing the observed levels to levels of AZI found in a normal control sample, for example a sample obtained from a patient that does not have cancer. Levels of AZI higher than levels that are observed in the normal control indicate the presence of cancer. The levels of AZI can be represented by arbritary units, for example as units obtained from a densitometer, luminometer, or an ELISA plate reader.

As used herein, “a higher level of AZI in the test sample as compared to the level in the normal control sample” refers to an amount of AZI that is greater than an amount of AZI present in a normal control sample. The term “higher level” refers to a level that is statistically significant or significantly above levels found in the normal control sample. Preferably, the “higher level” is at least 2 fold greater.

The term “statistically significant” or “significantly” refers to statistical significance and generally means a two standard deviation (2SD) above normal, or higher, concentration of the marker.

As used herein, “a high level” of AZI refers to amounts of AZI that are at least about 3 fold greater than the amounts of AZI present in normal control samples, preferably about 5 fold to about 6 fold greater.

For purposes of comparison, the test sample and normal control sample are of the same type, that is, obtained from the same biological source. The normal control sample can also be a standard sample that contains the same concentration of AZI that is normally found in a biological sample of the same type and that is obtained from a healthy individual. For example, there can be a standard normal control sample for the amounts of AZI normally found in biological samples such as urine, blood, cerebral spinal fluid, or tissue.

Additionally, disease progression can be assessed by following AZI levels in individual patients over time. Cancers include, for example, stomach cancer, lung cancer (see e.g. Bhattacharjee et al, (Proc Natl Acad Sci USA. 2001, 98(24):13790-5; Garber M E et al., Proc Natl Acad Sci USA. 2001, 98(24):13784-9); liver cancer (Chen et al., (Mol Biol Cell. 2002, 13(6):1929-39); brain cancer (Watson et al., Am. J. Pathol. 2002, 161(2):665-72; Gutmann et al., (Cancer Res. 2002, 62(7):2085-91); and adrenal cancer (Giordano et al., (Am J. Pathol. 2003, 162(2):521-31).

The present invention further provides for methods of prognostic evaluation of a patient suspected of having, or having, cancer. The method comprises measuring the level of AZI present in a test biological sample obtained from a patient and comparing the observed level with a range of AZI levels normally found in biological samples (of the same type) of healthy individuals. An increased level for example, is indicative of a greater potential for metastatic activity and corresponds to a poor prognosis. Higher levels also indicate that the tumor is more aggressive.

This information can be used by the physician in determining the most effective course of treatment. A course of treatment refers to the therapeutic measures taken for a patient after diagnosis or after treatment for cancer. For example, a determination of the likelihood for cancer recurrence, spread, or patient survival, can assist in determining whether a more conservative or more radical approach to therapy should be taken, or whether treatment modalities should be combined. For example, when cancer recurrence is likely, it can be advantageous to precede or follow surgical treatment with chemotherapy, radiation, immunotherapy, biological modifier therapy, gene therapy, vaccines, and the like, or adjust the span of time during which the patient is treated.

Changes in a patient's condition can be monitored using the methods of the present invention by comparing changes in AZI expression levels in the tumor in that subject over time.

Biological specimens include, for example, blood, tissue, serum, stool, urine, sputum, nipple aspirates, cerebrospinal fluid and supernatant from cell lysate. Preferably, one uses tissue specimens, serum or urine. The determination of, and comparison of, AZI levels is by standard modes of analysis based upon the present disclosure.

The methods of the invention can also be practiced, for example, by selecting a combination of AZI and one or more biomarkers for which increased or decreased expression correlates with cancer, such as any of thymosin β15 (See for example PCT publication WO 97/48805), thymosin β4, thymosin β10, cIAP2, Apaf1, Bcl-2, Smac, MMP-1, MMP-2, MMP-9, other MMPs, or another known or standard biomarker for cancer. The selected biomarker can be a general diagnostic or prognostic marker useful for multiple types of cancer, such as CA 125, CEA or LDH, or can be a cancer-specific diagnostic or prognostic marker, such as a colon cancer marker (for example, sialosyl-TnCEA, CA19-9, or LASA), breast cancer marker (for example, CA 15-2. Her-2/neu and CA 27.29), ovarian cancer marker (for example, CA72-4), lung cancer (for example, neuron-specific enolase (NSE) and tissue polypeptide antigen (TPA)), prostate cancer (for example, PSA, prostate-specific membrane antigen and prostatic acid phosphatase), melanoma (for example, S-100 and TA-90), as well as other biomarkers specific for other types of cancer. Those skilled in the art will be able to select useful diagnostic or prognostic markers for detection in combination with AZI. Similarly, three or more, four or more or five or more or a multitude of biomarkers can be used together for determining a diagnosis or prognosis of a patient.

### Antizyme Inhibitor Nucleic Acid Probes

Types of probe include cDNA, riboprobes, synthetic oligonucleotides and genomic probes. The type of probe used will generally be dictated by the particular situation, such as riboprobes for in situ hybridization, and cDNA for Northern blot hybridization analysis, for example. Most preferably, the probe is directed to nucleotide regions unique to the AZI sequence. Detection of the antizyme inhibitor encoding gene, per se, will be useful in screening for mutations associated with enhanced expression. Other forms of assays to detect targets more readily associated with levels of expression, transcripts and other expression products, will generally be useful as well. The probes may be as short as is required to differentially recognize antizyme inhibitor mRNA transcripts, and may be as short as, for example, 15 bases; however, probes of at least 17 bases, more preferably 18 bases and still more preferably 20 bases are preferred.

A probe may also be reverse-engineered by one skilled in the art from the amino acid sequence of the antizyme inhibitor. However use of such probes may be more limited than the native DNA sequence, as it will be appreciated that any one given reverse-engineered sequence will not necessarily hybridize well, or at all, with any given complementary sequence reverse-engineered from the same peptide, owing to the degeneracy of the genetic code. This is a factor common in the calculations of those skilled in the art, and the degeneracy of any given sequence is frequently so broad as to yield a large number of probes for any one sequence.

The form of labeling of the probes may be any that is appropriate, such as the use of radioisotopes, for example, ³²P and ³⁵S, and fluorescent labels Labeling with radioisotopes may be achieved, whether the probe is synthesized chemically or biologically, by the use of suitably labeled bases.

### Antizyme Inhibitor RNA Detection Techniques

Detection of RNA transcripts may be achieved by Northern blot hybridization, for example, wherein a preparation of RNA is run on a denaturing agarose gel, and transferred to a suitable support, such as activated cellulose, nitrocellulose or glass or nylon membranes. Radiolabeled cDNA or RNA is then hybridized to the preparation, washed and analyzed by autoradiography.

Detection of RNA transcripts can further be accomplished using known amplification methods. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994).

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 454-4610; strand displacement amplification (as described in G. T. Walker et al., Clin. Chem. 42: 9-13 (1996) and European Patent Application No. 684315; and target mediated amplification, as described by PCT Publication WO 9322461.

In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with haematoxylin to demonstrate the histological composition of the sample, and dark field imaging with a suitable light filter shows the developed emulsion. Non-radioactive labels such as digoxigenin may also be used.

Alternatively, mRNA expression can be detected on a DNA array, chip or a microarray. Oligonucleotides corresponding to the antizyme inhibitor are immobilized on a chip which is then hybridized with labeled nucleic acids of a test sample obtained from a patient. Positive hybridization signal is obtained with the sample containing antizyme inhibitor transcripts. Methods of preparing DNA arrays and their use are well known in the art. (See, for example U.S. Pat. Nos. 6,618,6796; 6,379,897; 6,664,377; 6,451,536; 548,257; U.S. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See for example U.S. Patent Application 20030215858).

To monitor mRNA levels, for example, mRNA is extracted from the biological sample to be tested, reverse transcribed, and fluorescent-labeled cDNA probes are generated. The microarrays capable of hybridizing to antizyme inhibitor cDNA are then probed with the labeled cDNA probes, the slides scanned and fluorescence intensity measured. This intensity correlates with the hybridization intensity and expression levels.

The present invention further provides additional diagnostic or prognostic methods, comprising methods having the patient tested. The test comprising the methods for determining or measuring AZI levels set forth above. The results are the reviewed by a clinician or the service provider and appropriate treatment is administered.

### Antizyme Inhibitor Antibodies

Antibodies may be raised against either a peptide of antizyme inhibitor or the whole molecule. For example, a peptide may be presented together with a carrier protein, such as an KLH, to an animal system or, if it is long enough, say 25 amino acid residues, without a carrier. Antibodies can also be raised against homologs or orthologs of an antizyme inhibitor. Modified antizyme inhibitor proteins may also be used, for example chemically modified proteins (e.g. methylation, acetylation, or others), fusion proteins, or mutants. All that is required is that the antibody produced specifically binds to antizyme inhibitor.

Polyclonal antibodies generated by the above technique may be used directly, or suitable antibody producing cells may be isolated from the animal and used to form a hybridoma by known means (Kohler and Milstein, Nature 256:795. (1975)). Selection of an appropriate hybridoma will also be apparent to those skilled in the art, and the resulting antibody may be used in a suitable assay to identify antizyme inhibitor.

The term “antibody” as used herein encompasses polyclonal or monoclonal antibodies as well as functional fragments of antibodies, including fragments of chimeric, human, humanized, primatized, veneered or single-chain antibodies. Functional fragments include antigen-binding fragments which bind to antizyme inhibitor. For example, antibody fragments capable of binding to antizyme inhibitor or portions thereof, including, but not limited to Fv, Fab, Fab′ and F (ab′) 2 fragments can be used. Such fragments can be produced by enzymatic cleavage or by recombinant techniques. For example, papain or pepsin cleavage can generate Fab or F (ab′) 2 fragments, respectively. Other proteases with the requisite substrate specificity can also be used to generate Fab or F (ab′) 2 fragments. Antibodies can also be produced in a variety of truncated forms using antibody genes in which one or more stop codons have been introduced upstream of the natural stop site. For example, a chimeric gene encoding a F (ab′) 2 heavy chain portion can be designed to include DNA sequences encoding the CH, domain and hinge region of the heavy chain.

Single-chain antibodies, and chimeric, human, humanized or primatized (CDR-grafted), or veneered antibodies, as well as chimeric, CDR-grafted or veneered single-chain antibodies, comprising portions derived from different species, and the like are also encompassed by the present invention and the term “antibody”. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies.

Preparation of Immunizing Antigen, and Polyclonal and Monoclonal Antibody production can be performed using any suitable technique. For example, monoclonal antibodies directed against binding cell surface epitopes can be readily produced by one skilled in the art. The general methodology for making monoclonal antibodies by hybridomas is well known. Other suitable methods of producing or isolating antibodies of the requisite specificity can be used, including, for example, methods which select recombinant antibody from a library (e.g., a phage display library).

In some embodiments, agents that specifically bind to AZI other than antibodies are used, such as peptides. Peptides that specifically bind to AZI can be identified by any means known in the art. For example, specific peptide binders of an AZI can be screened for using peptide phage display libraries.

For example, we have successfully raised a polyclonal antibody against AZI. The rabbit polyclonal antibody against AZI was raised against a C-terminal peptide (CIQLSQEDNFSTEA, SEQ ID NO.: 3) of rat AZI. The peptide was synthesized and conjugated to KLH (SynPep, Dublin, Calif.). Two rabbits were immunized with the antigen (Covance, Denver, Pa.). Affinity-purified rabbit polyclonal AZI antibody was obtained by passing the serum over a column with the full-length GST-tagged rat AZI protein crosslinked to glutathione-sepharose (Amersham Biosciences). This antibody works for immunoblot and immunofluorescence.

### Antizyme Inhibitor Protein Detection Techniques

It is generally preferred to use antibodies, or antibody equivalents, to detect antizyme inhibitor protein. Methods for the detection of protein are well known to those skilled in the art, and include ELISA (enzyme linked immunosorbent assay), RIA (radioimmunoassay), Western blothybridization, and immunohistochemistry. Immunoassays such as ELISA or RIA, which can be extremely rapid, are more generally preferred. Antibody arrays or protein chips can also be employed, see for example U.S. Patent Application Nos: 20030013208A1; 20020155493A1, 20030017515 and U.S. Pat. Nos. 6,329,209; 6,365,418, herein incorporated by reference in their entirety.

Samples for diagnostic purposes may be obtained from any number of sources. A sample obtained directly from the tumor, such as the stroma or cytosol, may be used to determine the metastatic potential of the tumor. It may also be appropriate to obtain the sample from other biological specimens, such as blood, lymph nodes, or urine. Such diagnosis may be of particular importance in monitoring progress of a patient, such as after surgery to remove a tumor. If a reference reading is taken after the operation, then another taken at regular intervals, any rise could be indicative of a relapse, or possibly a metastasis.

ELISA and RIA procedures may be conducted such that an antizyme inhibitor standard is labeled (with a radioisotope such as ¹²⁵I or ³⁵S, or an assayable enzyme, such as horseradish peroxidase or alkaline phosphatase), and, together with the unlabelled sample, brought into contact with the corresponding antibody, whereon a second antibody is used to bind the first, and radioactivity or the immobilized enzyme assayed (competitive assay). Alternatively antizyme inhibitor in the sample is allowed to react with the corresponding immobilized antibody, radioisotope- or enzyme-labeled anti-antizyme inhibitor antibody is allowed to react with the system, and radioactivity or the enzyme assayed (ELISA-sandwich assay). Other conventional methods may also be employed as suitable.

The above techniques may be conducted essentially as a “one-step” or “two-step” assay. The “one-step” assay involves contacting antigen with immobilized antibody and, without washing, contacting the mixture with labeled antibody. The “two-step” assay involves washing before contacting the mixture with labeled antibody. Other conventional methods may also be employed as suitable.

Enzymatic and radiolabeling of antizyme inhibitor and/or the antibodies may be effected by conventional means. Such means will generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary for all of the enzyme to be active, provided that enough remains active to permit the assay to be effected. Indeed, some techniques for binding enzyme are non-specific (such as using formaldehyde), and will only yield a proportion of active enzyme.

It is usually desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene may provide a suitable support.

Enzymes employable for labeling are not particularly limited, but may be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase may be assayed by measuring the concentration of hydrogen peroxide formed after reaction of the enzyme-labeled antibody with the substrate under controlled conditions well-known in the art.

Other techniques may be used to detect antizyme inhibitor according to a practitioner's preference based upon the present disclosure. One such technique is Western blothybridization (Towbin et at., Proc. Nat. Acad. Sci. 76:4350 (1979)), wherein a suitably treated sample is run on an SDS-PAGE gel before being transferred to a solid support, such as a nitrocellulose filter. antizyme inhibitor antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection may also be used.

Immunohistochemistry may be used to detect expression of antizyme inhibitor in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabelling. The assay is scored visually, using microscopy.

In addition, the antizyme inhibitor protein may be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In certain embodiments, a gas phase ion spectrophotometer is used. In other embodiments, laser-desorption/ionization mass spectrometry is used to analyze the sample. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes. See, e.g., U.S. Pat. No. 5,118,937 (Hillenkamp et al.), and U.S. Pat. No. 5,045,694 (Beavis & Chait).

In SELDI, the substrate surface is modified so that it is an active participant in the desorption process. In one variant, the surface is derivatized with adsorbent and/or capture reagents that selectively bind the protein of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied. See, e.g., U.S. Pat. No. 5,719,060 (Hutchens & Yip) and WO 98/59361 (Hutchens & Yip). The two methods can be combined by, for example, using a SELDI affinity surface to capture an analyte and adding matrix-containing liquid to the captured analyte to provide the energy absorbing material.

For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4.sup.th ed. Vol. 15 (John Wiley & Sons, New York 1995), pp. 1071-1094.

Detection of the presence of a marker or other substances will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of particular biomolecules. Software programs such as the Biomarker Wizard program (Ciphergen Biosystems, Inc., Fremont, Calif.) can be used to aid in analyzing mass spectra. The mass spectrometers and their techniques are well known to those of skill in the art.

Any person skilled in the art understands, any of the components of a mass spectrometer (e.g., desorption source, mass analyzer, detect, etc.) and varied sample preparations can be combined with other suitable components or preparations described herein, or to those known in the art. For example, in some embodiments a control sample may contain heavy atoms (e.g. ¹³C) thereby permitting the test sample to mixed with the known control sample in the same mass spectrometry run.

In one preferred embodiment, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

In some embodiments the relative amounts of one or more biomolecules present in a first or second sample is determined, in part, by executing an algorithm with a programmable digital computer. The algorithm identifies at least one peak value in the first mass spectrum and the second mass spectrum. The algorithm then compares the signal strength of the peak value of the first mass spectrum to the signal strength of the peak value of the second mass spectrum of the mass spectrum. The relative signal strengths are an indication of the amount of the biomolecule that is present in the first and second samples. A standard containing a known amount of a biomolecule can be analyzed as the second sample to provide better quantify the amount of the biomolecule present in the first sample. In certain embodiments, the identity of the biomolecules in the first and second sample can also be determined.

### Screening for New Treatment Agents

As described in the Examples, below, the AZI protein is localized to the centrosome in cells and participates in the proper segregation of chromosomes during mitosis. Disregulation of AZI results in multiple centrosomes and aneuploidy, both of which are hallmarks of cancer. Recent attention to centrosomal proteins as targets for cancer therapeutics has included the development of inhibitors for centrosome-localized kinases such as polo kinases and aurora kinase.

Accordingly, in addition to the use of known AZI inhibitors such as siRNA, as described above, the invention also provides methods to identify novel cancer therapeutic agents and compounds, by screening for agents which downregulate the expression of AZI or inhibit its activity.

One embodiment of the invention provides a method for screening for a compound or agent which modulates the expression of AZI which is useful for the treatment of cancer, comprising providing a cell comprising a reporter plasmid comprising the regulatory elements of the AZI gene, including the promoter and the 3′ untranslated region, functionally connected to a nucleic acid encoding a detectable protein; contacting the cell with a test compound or agent; detecting the level of expression of the reporter gene; comparing the level of reporter expression in the presence of the test compound with the level of reporter expression in a control sample in the absence of the test compound; and determining whether or not the test compound increases or decreases the level of reporter expression, wherein a decrease in the level of reporter expression indicates the compound or agent is an antizyme inhibitor.

Another embodiment of the invention provides an assay for screening for agents capable of inhibiting AZI, comprising the steps of providing a cell transfected with a vector comprising a nucleic acid sequence encoding AZI or a functional fragment thereof; providing an assay for functional mitosis; contacting said cell with a test agent; detecting the amount of functional mitosis in the sample; and comparing the amount of functional mitosis expression in the presence of the test compound with the level of functional mitosis in a control sample in the absence of the test compound; and determining whether or not the test compound increases or decreases the level of functional mitosis, wherein a decrease in the level of functional mitosis indicates the compound or agent is an antizyme inhibitor.

The invention provides efficient screening methods to identify pharmacological agents or lead compounds for agents that modulate, e.g. interfere with or increase AZI expression or activity. The methods are amenable to automated, cost-effective high throughput drug screening and have immediate application in a broad range of pharmaceutical drug development programs.

For example, in one embodiment of the invention, compounds are screened first for their ability to bind AZI and second for their ability to disrupt mitotic function.

A wide variety of assays for AZI binding agents are provided including, e.g., labelled in vitro protein-protein binding assays, electrophoretic mobility shift assays, immunoassays, cell based assays such as one, two and three hybrid screens and expression assays.

The present invention also provides assays to assess mitotic function, including the integrity of the centrosome. One such assay is determination of proliferation. Another such assay is localization of centrosomal proteins, including but not limited to gamma-tubulin and pericentrin, as an indicator of the integrity of the centrosome. Another such assay is the localization of AZI to the centrosome. Yet another assay is to count the number of centrosomes per cell. Yet another assay uses fluorescence-activated cell sorting (FACS) based in DNA distribution within a cell. Yet another Such assays include both microscopy based assays such as immunohistochemistry as well as biochemical assays such as cellular fractionation. These assays are well known in the art, and are described in detail in the Examples, below.

An assay mixture of the invention comprises at least a portion of the AZI protein. An assay mixture of the invention also comprises a candidate pharmacological agent. Generally a plurality of assay mixtures are run in parallel with different candidate agent concentrations to obtain a differential response to the various concentrations. Typically, one of these assay mixtures serves as a negative control, i.e. at zero concentration or below the limits of assay detection. Candidate agents encompass numerous chemical classes, though typically they are organic or inorganic compounds and preferably small organic compounds. Small organic compounds generally have a molecular weight of more than about 50 yet less than about 2,500. Candidate agents comprise functional chemical groups necessary for structural interactions with proteins and/or DNA, and may include at least one or two amine, carbonyl, hydroxyl or carboxyl groups.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced.

Additionally, natural and synthetically produced libraries and compounds are readily modified through conventional chemical, physical, and biochemical means. In addition, known pharmacological agents may be subject to directed or random chemical modifications, such as acylation, alkylation, esterification, amidification, etc.

A variety of other reagents may also be included in the mixture. These include reagents such as salts, buffers, neutral proteins, e.g. albumin, detergents, etc. which may be used to facilitate optimal protein-protein and/or protein-nucleic acid binding and/or reduce non-specific or background interactions, etc. Also, reagents that otherwise improve the efficiency of the assay, such as protease inhibitors, nuclease inhibitors, antimicrobial agents, etc. may be used.

The resultant mixture is then incubated under conditions whereby the candidate pharmacological agent and the AZI or fragment or derivative thereof, if capable, bind. The mixture components can be added in any order that provides for the requisite bindings. Incubations may be performed at any temperature that facilitates optimal binding, typically between 4° and 40° C., more commonly between 150 and 40° C. Incubation periods are likewise selected for optimal binding but also minimized to facilitate rapid, high throughput screening, and are typically between 0.1 and 10 hours, preferably less than 5 hours, more preferably less than 2 hours.

After incubation, the binding is detected by any convenient way. For cell-free type assays, the AZI may be bound to a solid substrate and the agent labeled, e.g., radiolabeled. A separation step can be used to separate the bound AZI from unbound agent. The separation step may be accomplished in a variety of ways known in the art. The solid substrate may be made of a wide variety of materials and in a wide variety of shapes, e.g. microtiter plate, microbead, dipstick, resin particle, etc. The substrate is chosen to maximize signal to noise ratios, to minimize background binding, to facilitate washing and to minimize cost.

Separation may be effected for example, by removing a bead or dipstick from a reservoir, emptying or diluting a reservoir such as a microtiter plate well, rinsing a bead (e.g. beads with iron cores may be readily isolated and washed using magnets), particle, chromatographic column or filter with a wash solution or solvent. Typically, the separation step will include an extended rinse or wash or a plurality of rinses or washes. For example, where the solid substrate is a microtiter plate, the wells may be washed several times with a washing solution, which typically includes those components of the incubation mixture that do not participate in specific binding such as salts, buffer, detergent, nonspecific protein, etc. may exploit a polypeptide specific binding reagent such as an antibody or receptor specific to a ligand of the polypeptide.

As mentioned, detection may be effected in any convenient way, and for cell-free assays, one of the components usually comprises or is coupled to a label. Essentially any label can be used that provides for detection. The label may provide for direct detection as radioactivity, luminescence, optical or electron density, etc. or indirect detection such as an epitope tag, an enzyme, etc. The label may be appended to a reagent or incorporated into the peptide structure, e.g. in the case of a peptide reagent, a methionine residue comprising a radioactive isotope of sulfur.

A variety of methods may be used to detect the label depending on the nature of the label and other assay components. For example, the label may be detected bound to the solid substrate or a portion of the bound complex containing the label may be separated from the solid substrate, and thereafter the label detected. Labels may be directly detected through optical or electron density, radiative emissions, nonradiative energy transfers, etc. or indirectly detected with antibody conjugates, etc. For example, in the case of radioactive labels, emissions may be detected directly, e.g. with particle counters or indirectly, e.g. with scintillation cocktails and counters.

The assays of the invention are particularly suited to automated high throughput drug screening. In a particular embodiment, an automated mechanism, e.g. a mechanized arm, retrieves and transfers a microtiter plate to a liquid dispensing station where measured aliquots of each of an incubation buffer and a solution comprising one or more candidate agents are deposited into each designated well. The arm then retrieves and transfers to and deposits in designated wells a measured aliquot of a solution comprising an AZI protein or fragment or derivative thereof as well as solutions of other reagents. Thereafter, the arm transfers the microtiter plate to an analysis station where the reaction mixture can be analyzed for the presence or absence of binding.

In one embodiment the invention provides antibodies against AZI proteins or antigenic fragments thereof. Such antibodies can readily be obtained by using antigenic portions of the protein to screen an antibody library such as a phage display library. Antibodies also can be prepared that will bind to one or more particular domains of a peptide of the invention and can be used to modulate AZI activity.

### Administration

Pharmaceutical compositions of the present invention may be administered either prior to or following surgical removal of primary tumors and/or treatment such as administration of radiotherapy or conventional chemotherapeutic drugs. As discussed below, administration of the pharmaceutical compositions may be by any suitable method, including administration by intravenous, intraperitoneal, intramuscular, subcutaneous, intranasal, intradermal, anal, vaginal, topical and oral routes.

The term “pharmaceutically acceptable” refers to compounds and compositions which may be administered to mammals without undue toxicity. Exemplary pharmaceutically acceptable salts include mineral acid salts such as hydrochlorides, hydrobromides, phosphates, sulfates, and the like; and the salts of organic acids such as acetates, propionates, malonates, benzoates, and the like.

The compounds and agents of the invention are administered orally, topically, or by parenteral means, including subcutaneous and intramuscular injection, implantation of sustained release depots, intravenous injection, intranasal administration, and the like. Accordingly, agents of the invention may be administered as a pharmaceutical composition comprising the agonist or antagonist in combination with a pharmaceutically acceptable carrier. Such compositions may be aqueous solutions, emulsions, creams, ointments, suspensions, gels, liposomal suspensions, and the like. Suitable carriers (excipients) include water, saline, Ringer's solution, dextrose solution, and solutions of ethanol, glucose, sucrose, dextran, mannose, mannitol, sorbitol, polyethylene glycol (PEG), phosphate, acetate, gelatin, collagen, Carbopol®, vegetable oils, and the like. One may additionally include suitable preservatives, stabilizers, antioxidants, antimicrobials, and buffering agents, for example, BHA, BHT, citric acid, ascorbic acid, tetracycline, and the like. Cream or ointment bases useful in formulation include lanolin, Silvadene® (Marion), Aquaphor® (Duke Laboratories), and the like. Other topical formulations include aerosols, bandages, and other wound dressings. Alternatively one may incorporate or encapsulate the compounds in a suitable polymer matrix or membrane, thus providing a sustained-release delivery device suitable for implantation near the site to be treated locally. Other devices include indwelling catheters and devices such as the Alzet® minipump. Ophthalmic preparations may be formulated using commercially available vehicles such as Sorbi-care® (Allergan), Neodecadron® (Merck, Sharp & Dohme), Lacrilube®, and the like, or may employ topical preparations such as that described in U.S. Pat. No. 5,124,155, incorporated herein by reference. Further, one may provide an antagonist in solid form, especially as a lyophilized powder. Lyophilized formulations typically contain stabilizing and bulking agents, for example human serum albumin, sucrose, mannitol, and the like. A thorough discussion of pharmaceutically acceptable excipients is available in Remington's Pharmaceutical Sciences (Mack Pub. Co.).

The amount of agent required to treat any particular disorder will of course vary depending upon the nature and severity of the disorder, the age and condition of the subject, and other factors readily determined by one of ordinary skill in the art. Routes and frequency of administration, as well as dosage, will vary from individual to individual.

The percentage of the agents in the composition is about 0.01 wt. % to about 10 wt. %, and preferably, about 0.01 wt. % to about 2 wt. %.

The topical compositions and drug delivery systems of the invention can be used in the prevention or treatment of the cancers described above. In treating cancer, it will be recognized by those skilled in the art that the optimal quantity and spacing of individual dosages will be determined by the nature and extent of the condition being treated, the form, route and site of administration, and the particular individual undergoing treatment, and that such optimums can be determined by conventional techniques. It will also be appreciated by one skilled in the art that the optimal dosing regimen, i.e., the number of doses can be ascertained using conventional course of treatment determination tests. Generally, a dosing regimen will involve administration of the selected topical formulation at least once daily, and preferably one to four times daily, until the symptoms have subsided.

Administration of a composition may be by systemic route, including oral, parenteral, sublingual, rectal such as suppository or enteral administration, or by pulmonary absorption, or by topical administration. Parenteral administration may be by intravenous injection, subcutaneous injection, intramuscular injection, intra-arterial injection, intrathecal injection, intra peritoneal injection or direct injection or other administration to one or more specific sites. When long term administration by injection is necessary, venous access devices such as medi-ports, in-dwelling catheters, or automatic pumping mechanisms are also preferred wherein direct and immediate access is provided to the arteries in and around the heart and other major organs and organ systems.

Compositions may also be administered to the nasal passages as a spray. Arteries of the nasal area provide a rapid and efficient access to the bloodstream and immediate access to the pulmonary system. Access to the gastrointestinal tract, which can also rapidly introduce substances to the blood stream, can be gained using oral enema, or injectable forms of administration. Compositions may be administered as a bolus injection or spray, or administered sequentially over time (episodically) such as every two, four, six or eight hours, every day (QD) or every other day (QOD), or over longer periods of time such as weeks to months. Compositions may also be administered in a timed-release fashion such as by using slow-release resins and other timed or delayed release materials and devices.

Where systemic administration is desired, orally active compositions are preferred as oral administration is a convenient and economical mode of drug delivery. Oral compositions may be poorly absorbed through the gastrointestinal lining. Compounds which are poorly absorbed tend to be highly polar. Preferably, such compositions are designed to reduce or eliminate their polarity. This can be accomplished by known means such as formulating the oral composition with a complimentary reagent which neutralizes its polarity, or by modifying the compound with a neutralizing chemical group. Preferably, the molecular structure is similarly modified to withstand very low pH conditions and resist the enzymes of the gastric mucosa such as by neutralizing an ionic group, by covalently bonding an ionic interaction, or by stabilizing or removing a disulfide bond or other relatively labile bond.

Treatments to the patient may be therapeutic or prophylactic. Therapeutic treatment involves administration of one or more compositions of the invention to a patient suffering from one or more symptoms of the disorder. Relief and even partial relief from one or more symptoms can correspond to an increased life span or simply an increased quality of life. Further, treatments that alleviate a pathological symptom can allow for other treatments to be administered.

The term “compatible”, as used herein, means that the components of the compositions are capable of being commingled with the thyroid hormone conversion inhibitors of the present invention, and with each other, in a manner such that does not substantially impair the desired efficacy.

Doses of the pharmaceutical compositions of the invention will vary depending on the subject and upon the particular route of administration used. Dosages can range from 0.1 to 100,000 μg/kg per day, more preferably 1 to 10,000 μg/kg. By way of an example only, an overall dose range of from about, for example, 1 microgram to about 300 micrograms might be used for human use. This dose can be delivered at periodic intervals based upon the composition.

The system of the invention may be used advantageously with other treatment regiments. For example, the system may be used in conjunction with traditional treatment options for cancer including surgery, radiation therapy, chemotherapy, acupuncture, and acupressure.

Chemotherapy protocols for the treatment of a range of cancers are well known in the art, and can include a range of chemotherapeutic agents. One preferred group of chemotherapeutic agents are capecitabine, irinotecan or 5-fluorouracil. Other preferred chemotherapeutic agents are docetaxel, and gemcitabine.

Additional chemotherapeutic agents include the pharmaceutically acceptable taxanes, such as e.g. docetaxel, taxotere, Paclitaxel, 7-Epi-Taxol, 10-Deacetyl Taxol, as well as mixtures thereof, 5-fluorouracil (5-FU), cisplatin, gemcitabine, irinotecan (also called CPT-11), and tamoxifen. 5-FU may be administered with leucovorin. The chemotherapy can comprise doses of 5-FU ranging from 50 to 1000 mg/m.sup.2/d, with leucovorin at 90 mg/d to 100 mg/d or irinotecan ranging from 200-300 mg/m.sup.2/d, gemcitabine ranging from 100-1500 mg/m.sup.2/d; cisplatin (platinol) ranging from 40 mg-100 mg/m.sup.2/d; and tamoxifen from 10 mg-20 mg tablet per day. For example, combinations of chemotherapeutic agents comprise 5-FU Cisplatin, 5-FU-Gemcitabine or 5-FU with leucovorin & cisplatin.

Other examples of anti-cancer drugs that may be used in the various embodiments of the invention, including pharmaceutical compositions and dosage forms and kits of the invention, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine, mechlorethamine oxide hydrochloride rethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, improsulfan, benzodepa, carboquone, triethylenemelamine, trietylenephosphoramide, triethylenethiophosphaoramide, trimethylolomelamine, chlornaphazine, novembichin, phenesterine, trofosfamide, estermustine, chlorozotocin, gemzar, nimustine, ranimustine, dacarbazine, mannomustine, mitobronitol, aclacinomycins, actinomycin F(1), azaserine, bleomycin, carubicin, carzinophilin, chromomycin, daunorubicin, daunomycin, 6-diazo-5-oxo-1-norleucine, doxorubicin, olivomycin, plicamycin, porfiromycin, puromycin, tubercidin, zorubicin, denopterin, pteropterin, 6-mercaptopurine, ancitabine, 6-azauridine, carmofur, cytarabine, dideoxyuridine, enocitabine, pulmozyme, aceglatone, aldophosphamide glycoside, bestrabucil, defofamide, demecolcine, elformithine, elliptinium acetate, etoglucid, flutamide, hydroxyurea, lentinan, phenamet, podophyllinic acid, 2-ethylhydrazide, razoxane, spirogermanium, tamoxifen, taxotere, tenuazonic acid, triaziquone, 2,2′,2″-trichlorotriethylamine, urethan, vinblastine, vincristine, vindesine and related agents. 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; taxel; taxel analogues; taxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin. Additional cancer therapeutics include monoclonal antibodies such as rituximab, trastuzumab and cetuximab.

The magnitude of a prophylactic or therapeutic dose of each active ingredient in the treatment of a patient with a solid tumor will typically vary with the specific active ingredients, the severity and type of tumor, and the route of administration. The dose and the dose frequency may vary according to age, body weight, response, and the past medical history of the patient; the likelihood of mestastic recurrence must also be considered. Suitable dosing regimens can be readily selected by those skilled in the art with due consideration of such factors by following, for example, dosages reported in the literature and recommended in the Physician's Desk Reference® (54th ed., 2000). Unless otherwise indicated, the magnitude of a prophylactic or therapeutic dose of each pharmaceutical used in an embodiment of the invention will be that which is known to those in the art to be safe and effective, or is regulatory approved.

All references cited throughout the specification are herein incorporated by reference in their entirety.

The present invention is further illustrated by the following Examples. These Examples are provided to aid in the understanding of the invention and are not construed as a limitation thereof.

### EXAMPLES

Antizyme 1 is best known as a facilitator of ubiquitin-independent protein degradation. Overexpression of antizyme 1 promotes ODC degradation, inhibits ODC activity, suppresses cell proliferation and leads to apoptosis in several cell culture models (Koike et al., 1999; Iwata et al., 1999; Murakami et al., 1994; Tsuji et al., 2001). Further studies employing multiple in vivo models support the hypothesis that antizyme 1 inhibits tumour growth (Fong et al., 2003; Feith et al., 2001; Iwata et al., 1999; Tsuji et al., 2001). In addition, it has been reported recently that antizyme 1 has additional binding partners besides ODC and AZI and that it can promote degradation of cell cycle regulatory proteins such as Smad1 and cyclin D1 (Newman et al., 2004; Lin et al., 2002). In contrast to antizyme 1, the endogenous inhibitor AZI is associated with increased cell proliferation. AZI is rapidly induced in growth-stimulated mouse fibroblasts and it is upregulated in certain forms of human cancers, suggesting a possible role for AZI in tumorigenesis and cell cycle progression (Jung et al., 2000; Nilsson et al., 2000). Overexpression of ODC occurs in most forms of human malignancies (Gerner et al., 2004) and ODC is markedly induced in human prostate cancer (Mohan et al., 1999). In addition, certain tumour cells such as osteosarcoma cells are known to respond to inhibition of the polyamine biosynthesis pathway (Satoh et al., 1999). Thus, disregulation of antizyme 1 or AZI expression in prostate cancer or osteosarcoma cells, which are used here in this study, may facilitate the development of tumours in these tissues

### Material and Methods

### Cell Culture

Primary HFF (human foreskin fibroblasts) were prepared as described previously (Hasskarl et al (2004), Oncogene, 23(10), 1930-8). HFF and NIH/3T3 cells were maintained in DMEM with 10% BCS supplemented with 1% GPS (glutamine (2 mM), penicillin (50 U/ml) and streptomycin (50 ug/ml), Invitrogen). AT2.1 cells derived from the Dunning rat prostate carcinoma (John Isaacs, John Hopkins University) were maintained in RPMI media supplemented with 10% FBS, 1% GPS and 250 nM dexamethasone (Sigma, St. Louis, USA). U2OS cells were grown in DMEM, 10% FBS and 1% GPS. Human umbilical vascular endothelial cells (HUVEC) were purchased from Cambrex and maintained in EGM BulletKit medium (Cambrex) between passage 3 and 7.

### Immunofluorescence

For immunofluorescence microscopy, coverslips were coated with 10 μg/ml fibronectin (BD Biosciences, Bedford, Mass.) for 1 h at RT and washed with PBS. Cells were plated at subconfluent density and allowed to attach and spread onto the coverslips overnight. Cells were fixed in ice cold methanol for 10 min at −20° C., and permeabilized with 0.25% Triton-X 100 (Sigma, St. Louis, Mo.) or 0.05% Tween (Bio-Rad, Hercules, Calif.) in PBS for 15 min at RT. Samples were blocked in 1% BSA/PBS for 30 min and then stained with the following primary antibodies diluted in 1% BSA/PBS: mouse monoclonal AZI 1:1000 (gift of Dr. S. Matsufuji (Murakami et al., 1989)), rabbit polyclonal AZI 1:200 (affinity purified), rabbit polyclonal antizyme 1 serum 1:25 (gift of Dr. J. Mitchell), mouse monoclonal γ tubulin 1:400 (GTU-88; Sigma), rabbit polyclonal pericentrin 1:500 (Covance, Berkeley, Calif.), mouse monoclonal acetylated □-tubulin 1:200 (6-11B-1; Zymed, San Francisco, Calif.). All secondary antibodies were Alexa Fluor 488 or 568 conjugated goat anti-mouse or anti-rabbit 1:500 (Molecular Probes). Coverslips were washed and then mounted onto Vectashield mounting medium containing DAPI (Vector Laboratories, Burlingame, Calif.). IF microscopy was performed at RT using a Nikon Eclipse TE200 inverted microscope with epi-fluorescence attachment (Nikon, Melville, N.Y.). Images were captured using a SPOT RT camera, controlled by SPOT RT-Software v3.1 (Diagnostic Instruments, Sterling Heights, Mich.).

### Cell Culture and Transfection

Primary HFF (human foreskin fibroblasts) were prepared as described previously (Hasskarl et al., 2004). HFF and NIH-3T3 cells were maintained in DMEM (Invitrogen, Carlsbad, Calif.) with 10% bovine calf serum (BCS) supplemented with 1% GPS (glutamine (2 mM), penicillin (50 U/ml) and streptomycin (50 μg/ml), Invitrogen). U2OS cells were grown in DMEM with 10% FBS and 1% GPS. AT2.1 cells derived from the Dunning rat prostate carcinoma (John Isaacs, John Hopkins University (Isaacs et al., 1986)) were maintained in RPMI media supplemented with 10% FBS, 1% GPS and 250 nM dexamethasone (Sigma, St. Louis, USA). Human umbilical vein endothelial cells (HUVECs; Cambrex, Walkersville, Md.) were maintained in EGM medium (Cambrex, Walkersville, Md.) supplemented according to the manufacturer's instructions. All cells were grown at 37° C. and 5% carbon dioxide.

AT2.1, U2OS and NIH-3T3 cells were transiently transfected with siRNA using Lipofectamine 2000 (Invitrogen, Carlsbad, Calif.) according to the manufacturer's instructions. Stable transfectants expressing the pEGFP constructs were generated with Lipofectamine 2000. U2OS cells were selected in G418 (700 μg/ml) for two weeks. Single clones were isolated, expanded and analysed for GFP, GFP-antizyme or GFP-AZI expression.

siRNA Treatment

siRNAs targeting AZI or antizyme 1 and a non-specific control siRNA (control IX or scramble II Duplex, Dharmacon, Chicago, Ill.) were synthesized as complimentary single-stranded 19mer siRNAs and provided in the 2′-deprotected, duplexed, purified and desalted form. The siRNAs were delivered into cells using Lipofectamine 2000 (Invitrogen, Carlsbad, Calif.). The siRNAs targeting AZI were either rat/mouse nucleotide sequence 139-159 (AZI-139) or AZI Smartpool (consisting of four pooled SMARTselection-designed siRNAs against human, mouse or rat AZI, Dharmacon). The siRNAs targeting rat/mouse antizyme 1 were sequence 247-267 (AZ-1) and 264-284 (AZ-2).

### Generation of Constructs

Rat antizyme 1 was subcloned from pFLAG-AZ (Newman et al., 2004) into plasmid pEGFP-C3 (BD Biosciences, Mountain View, Calif.) using HindIII and EcoRI. Human AZI was amplified from pJG4-5-AZI (Mangold et al., 2005) and cloned into the XhoI and EcoRI sites of pEGFP-C1 (BD Biosciences) using GFP-AZI forward primer 5′-GGACTCGAGCTATGAAAGG ATTTATTGATGATGCAAAC-3′ (SEQ ID NO.: 1) and GFP-AZI reverse primer 5′-GCAGAATTCTTAA GCTTCAGCGGAAAAGCTG-3′ (SEQ ID NO.: 2). Both constructs were verified by multiple sequencing. Rat AZI was amplified from a rat liver cDNA library (gift Dr. M Klagsbrun, Children's Hospital Boston) and cloned into the GST-expression vector pGEX-2T (Amersham Biosciences, Piscataway, N.J.).

### Antibodies and Immunoblot Analysis

Protein samples were separated by 10% SDS-PAGE, transferred to nitrocellulose membrane (Protran, Schleicher & Schuell, Keene, N.H.) and blocked with 8% non-fat dry milk in PBS containing 0.1% Tween (PBST) for 1 h. The primary and secondary antibodies were diluted in PBST containing 5% milk and incubated for 1 h at RT. Primary antibodies used were antizyme rabbit serum 1:1000 (gift of Dr J. Mitchell); AZI mouse monoclonal 1:2000 (gift of Dr. S. Matsufuji), GFP rabbit serum 1:1000 (Molecular Probes, Eugene, Oreg.), γ-tubulin 1:2000 (GTU-88; Sigma), cyclin D1 1:250 (G124-326; BD Pharmingen), aurora A 1:500 (35C1; Abcam, Cambridge, Mass.), actin 1:10000 (C4; Chemicon, Temecula, Calif.). Secondary antibodies were peroxidase-conjugated anti-rabbit or anti-mouse 1:10000 (Pierce). The blots were developed with the Western Lightning™ Chemiluminescence Plus reagent (Perkin Elmer Life Science, Boston, Mass.) and exposed to Hyperfilm CDL (Amersham Biosciences, Piscataway, N.J.). Immunoblots were quantified using Scion Image Beta 4.02 software (Scion Corporation, Frederick, Md.).

The rabbit polyclonal antibody against AZI was raised against a C-terminal peptide (CIQLSQEDNFSTEA, SEQ ID NO.: 3) of rat AZI. The peptide was synthesized and conjugated to KLH (SynPep, Dublin, Calif.). Two rabbits were immunized with the antigen (Covance, Denver, Pa.). Affinity-purified rabbit polyclonal AZI antibody was obtained by passing the serum over a column with the full-length GST-tagged rat AZI protein crosslinked to glutathione-sepharose (Amersham Biosciences).

### Immunofluorescence (IF) Microscopy

For IF microscopy, coverslips were coated with 10 μg/ml fibronectin (BD Biosciences, Bedford, Mass.) for 1 h at RT and washed with PBS. Cells were plated at subconfluent density and allowed to attach and spread onto the coverslips overnight. Cells were fixed in ice cold methanol for 10 min at −20° C., and permeabilized with 0.25% Triton-X 100 (Sigma, St. Louis, Mo.) or 0.05% Tween (Bio-Rad, Hercules, Calif.) in PBS for 15 min at RT. Samples were blocked in 1% BSA/PBS for 30 min and then stained with the following primary antibodies diluted in 1% BSA/PBS: mouse monoclonal AZI 1:1000 (gift of Dr. S. Matsufuji (Murakami et al., 1989)), rabbit polyclonal AZI 1:200 (affinity purified), rabbit polyclonal antizyme 1 serum 1:25 (gift of Dr. J. Mitchell), mouse monoclonal γ-tubulin 1:400 (GTU-88; Sigma), rabbit polyclonal pericentrin 1:500 (Covance, Berkeley, Calif.), mouse monoclonal acetylated γ-tubulin 1:200 (6-1 lB-1; Zymed, San Francisco, Calif.). All secondary antibodies were Alexa Fluor 488 or 568 conjugated goat anti-mouse or anti-rabbit 1:500 (Molecular Probes). Coverslips were then mounted onto Vectashield mounting medium containing DAPI (Vector Laboratories, Burlingame, Calif.). IF microscopy was performed using a Nikon Eclipse TE200 inverted microscope with epi-fluorescence attachment (Nikon, Melville, N.Y.). Images were captured using a SPOT RT camera, controlled by SPOT RT-Software v3.1 (Diagnostic Instruments, Sterling Heights, Mich.).

### Competition Study Using Purified Recombinant Proteins

GST or GST-tagged rat AZI were expressed in E. coli strain BL21(DE3) using plasmid pGEX-AZI and purified with glutathione sepharose resin (Amersham Biosciences, Piscataway, N.J.) according to the manufacturer's instructions. For the competition study, cells were plated on coverslips and fixed in methanol. GST or GST-AZI (5 □g in 1% BSA/PBS) was added in combination with the primary antibody.

### Isolation of Centrosomes

Centrosomes were isolated from U2OS cells or U2OS cells stably overexpressing GFP-centrin as described (Mitchison et al., 1986; Bomens et al., 1987). The sucrose gradient fractions were analysed by immunoblot using antibodies against AZI, antizyme 1, γ-tubulin or GFP respectively.

### Aurora A Kinase Assay

Aurora A kinase activity was assayed with a non-radioisotopic kit for measuring aurora-A kinase activity (CycLex®, Nagano, Japan) following the manufacturer's instructions. 100 μg of total protein lysate were used per reaction.

### Statistical Methods

The mean percentage and standard deviation (s.d.) of two to three independent experiments with 100-200 cells evaluated per experiment are given unless indicated otherwise. Two-tailed student's t-test was used where applicable.

### Results and Discussion

Antizyme (AZ) is a protein that is known to modulate both the activity and the ubiquitin-independent degradation of the enzyme ornithine decarboxylase. Polyamines like putrescine are essential for cellular growth and differentiation but enhanced ODC activity is associated with cell transformation and cancer. More recently, it has been shown that AZ can associate with additional proteins such as Smad1, Snip1 and Cyclin D1 and can promote their degradation in vitro. AZ activity is known to be negatively regulated through the antizyme inhibitor (AZI), which binds to AZ with high affinity and releases ODC from the AZ-ODC complex. Whether AZI possesses other functions has not yet been determined. To further explore the role of AZ and AZI in cell cycle regulation, we studied AZ and AZI-Cyclin D1 interactions. We used siRNA to modulate the endogenous levels of AZ and AZI and investigated its effect on the stability of cell cycle proteins. Using pulse chase, we also studied the degradation of cycle D1 and a cyclin D1 mutant (T286A), which is not degraded via the ubiquitin pathway.

We showed the interaction of AZ with Cyclin D1. HEK 293 cells were transiently transfected with HA-cyclin D1 and FLAG-AZ. For immunoprecipitation, total cell extracts were incubated with an antibody against the FLAG epitope of AZ. Complex formation was assessed by immunoblot analysis using an antibody against the HA epitope of cyclin D1 (α-HA-CD1).

AZ binds to Cyclin D1 and promotes its degradation through the 26S proteasome. In the presence of AZI this degradation might be prevented.

We showed that overexpression of AZ leads to the induction of AZI and decreases Cyclin D1 levels. Dunning rat prostate carcinoma cells (AT2.1) and rat hepatoma cells (HTC) were transiently transfected with AZ (+FLAG-AZ) or the corresponding control vector (—FLAG-AZ). Expression of AZ, AZI Cyclin D1 and actin was determined by Western blot. (Newman et al, J. Biol. Chem., October 2004; 279: 41504-41511)

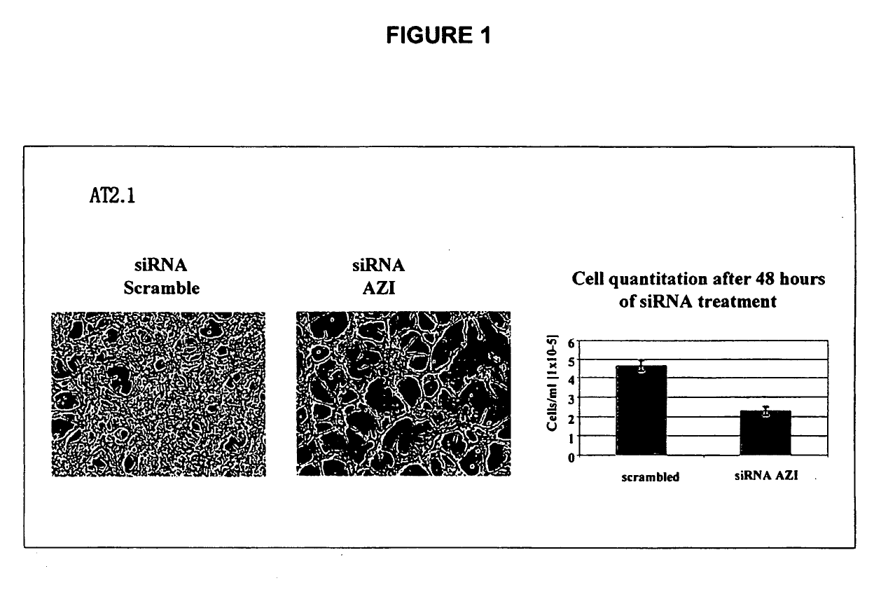

FIG. 1 shows that siRNA against AZI effects growth and cell shape of AT2.1 cells. AT2.1 cells were transiently transfected with siRNA against AZI or a scrambled control (scramble II Duplex, Dharmacon). Phase pictures were taken two days after transfection. Cells were quantified using a Coulter cell counter.

We showed that knock-down of AZI destabilizes D-type cyclins. HTC cells were transiently transfected with siRNA against AZI or with the corresponding control (scramble II Duplex, Dharmacon). Cells were incubated under normal growth conditions and gene silencing was monitored 48 h after transfection. Expression of AZI, D-type cyclins, CDK4, Cyclin A and actin was checked by Western blot hybridization.

We performed pulse-chase analysis of wildtype and mutant T26A Cycle D1. a-n, HTC cells were transiently transfected with pCMV-Flag or with pCMV-FlagAZ. 24 hours after transfection, cells were labeled with ³⁵S for 30 minutes prior to addition of chase media. Lysates were prepared 0, 10, 20, 40, 60, and 80 minutes after addition of chase media and the amount of cyclin D1 and AZ remaining at each time point was assessed by immunoprecipitation, SDS-PAGE, and phosphorimager analysis. E-d, HTC cells were transiently transfected with T286A mutant cyclin D1 along with either pCMV-Flag or with pCMV-FlagAZ. Pulse-chase experiment was carried out with using routine methods.

There are different substrate targeting pathways for the 26S proteasome. Schematic representation of the different targeting pathways: exclusively ubiquitin-dependent (B) ubiquitin and AZ-mediated (C) exclusively AZ-dependent (mammalian ODC).

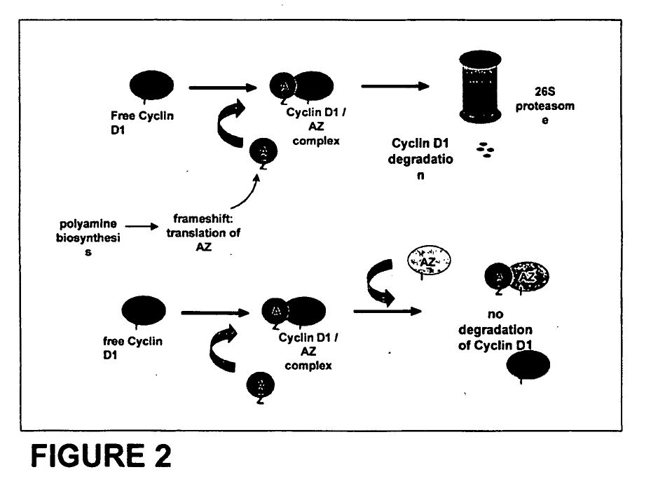

FIG. 2 is a schematic that summarizes the interation of ODC, AZ, and AZI. ODC, the rate limiting enzyme of polyamine biosynthesis, is active as a homodimer. ODC is negatively regulated through antizyme (AZ, which binds the ODC monomer and promotes degradation of ODC through the 26S proteasome. In addition, AZ also inhibits polyamine uptake. AZ targets proteins for degradation without ubiquitination. There are two different substrate targeting pathways for the 26S proteosome: an ubitquitin-dependent pathway for poly-ubiquitinated proteins, and an antizyme-dependent pathway for ODC (Murakami et al., (1992), Nature, 360(6404):597-9). AZ is in turn negatively regulated by the antizyme inhibitor (AZI), which binds AZ with higher affinity than ODC. AZI lacks PEST domains and is not targeted to the 26S proteasome by AZ.

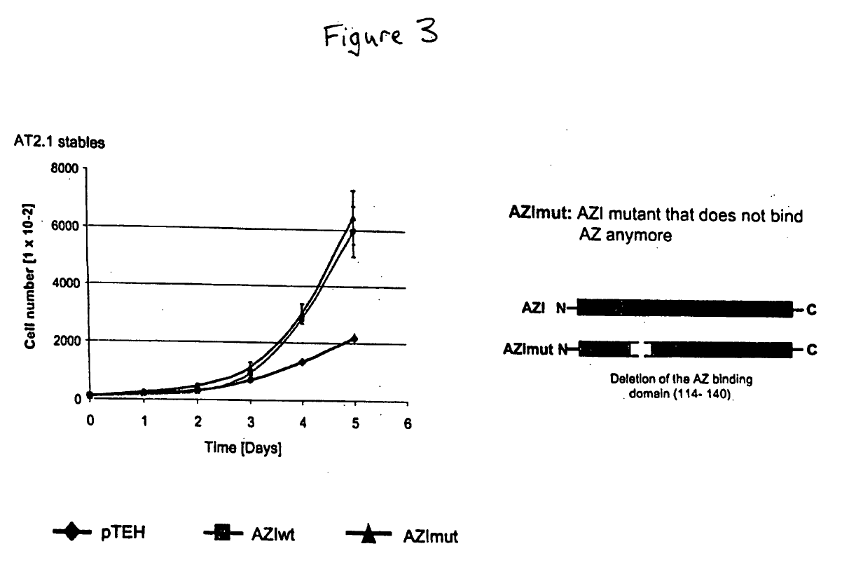

FIG. 3 shows that the effect of AZI overexpression on proliferation is not dependent on the activation of ODC. AT2.1 cells were transfected with either a control vector, pTEH (diamonds), the vector carrying the gene encoding wildtype AZI (AZIwt, shown as squares), or an AZI mutant that does not bind AZ (AZImut, shown as triangles), due to a deletion of the region encoding amino acids 114-140. Stable transfectants were selected and cells were grown for five days, with growth measures at each day. The overexpression of AZI in AT2.1 cells was associated with an increased rate of proliferation compared to the control cells; this effect on proliferation was not dependent on the ability of AZI to bind AZ.

AZI expression is elevated in cells, which overexpress H-ras, Human mammary epithelial cells (HMLE) which stably express either low levels or high levels of H-ras were compared for the expression of AZI, ras, and actin (control). Cells which expressed high levels of ras also expressed high levels of AZI, compared to cells which expressed lower levels of ras. Similarly, NIH/3T3 cells which were transiently transfected with H-ras expressed higher levels of AZI.

Human prostate carcinoma lines were analyzed for the expression of AZI. Three variants of the prostate carcinoma cell line PC3 were compared for expression levels of AZI. The PC3 cell line itself causes tumors in mice but does not metastasize. The PC-3M cell line forms tumors in mice and can be metastatic in mice. The PC-3M-LN4 cell line is the most metastatic variant, with he highest rates of both tumor formation and metastasis in mice. Thus, these three cell lines represent an increasing degree of metastatic potential. The expression of AZI was found to inversely correlate with the metastatic potential of the cell line, such that in the PC3M-LN4 cell line wildtype AZI levels are reduced, whereas a shorter variant of AZI is increased. We also performed an analogous experiments with the human prostate carcinoma cell line LnCap. Again, the more metatstatic potential a cell has, the less wildtype AZI it was found to express.

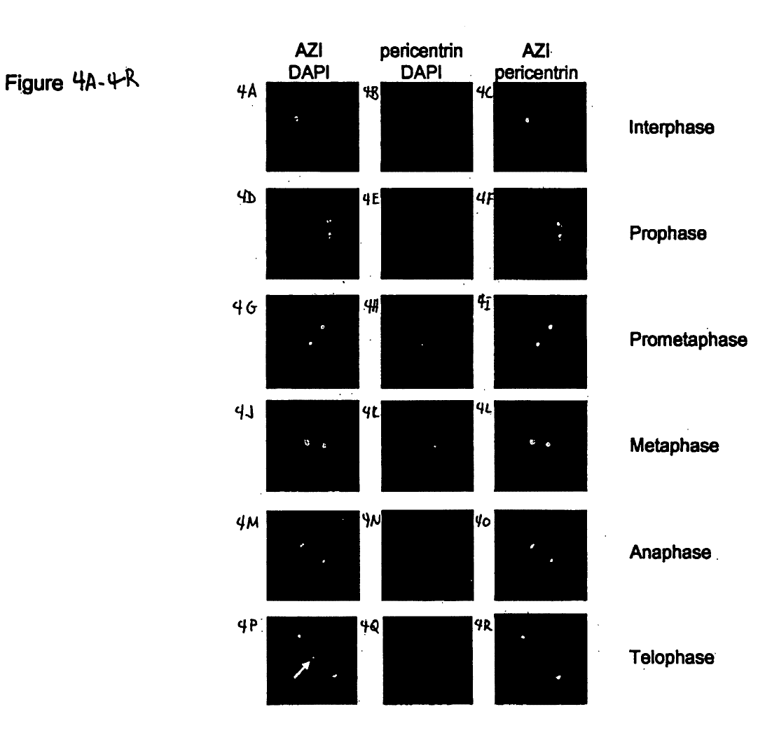

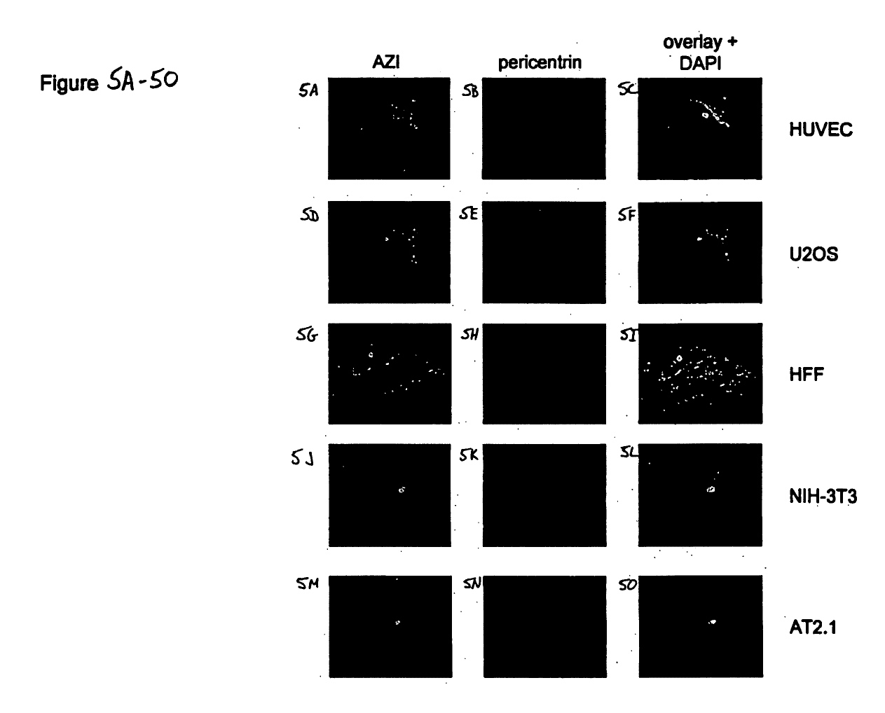

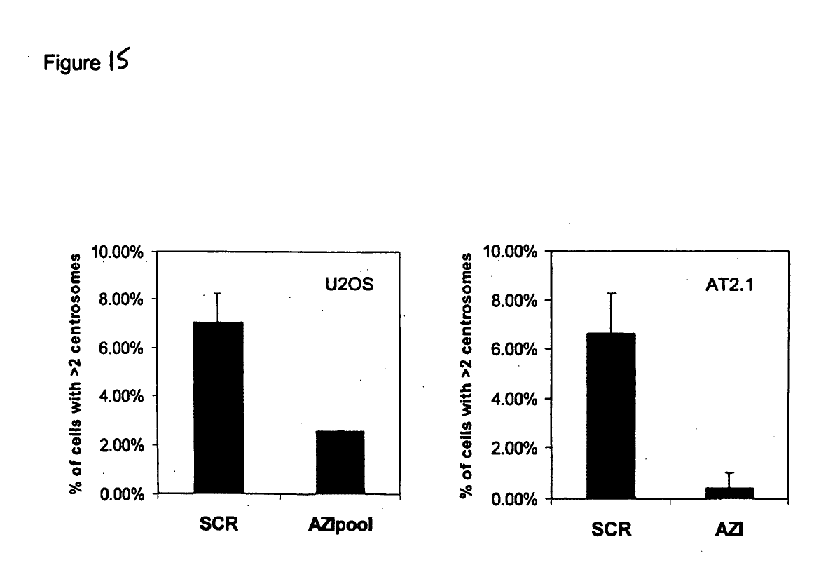

To further explore the functions of AZI, its cellular localization was determined by immunofluorescence using a mouse monoclonal antibody against AZI. FIG. 14 shows that AZI localizes to the centrosome. The chromosomal localization of AZI during cell cycle is shown in FIGS. 4A-4R. AZI localizes to centrosomes throughout the cell division cycle. Immunofluorescence analysis of non synchronized NIH-3T3 cells stained with antibodies against AZI (mouse monoclonal) and pericentrin (rabbit polyclonal). Nuclei were stained with DAPI. During telophase, an AZI signal also appeared adjacent to the intercellular bridge (white arrow).

FIGS. 5A-5O show the centrosomal localization of AZI during mitosis in U-2 OS cells fixed with formaldehyde. AZI localizes to centrosomes in different mammalian cell lines. Immunofluorescence analysis of AZI in human primary cells (HUVEC, HFF) or U2OS (human), AT2.1 (rat) and NIH-3T3 (mouse) cell lines. Centrosomes were visualized using a rabbit polyclonal antibody against pericentrin. AZI was visualized using the mouse monoclonal antibody. Nuclei were stained with DAPI.

We showed that a mouse monoclonal antibody raised against AZI shows specific immunofluorescence and centrosome staining. For example, immunofluorescence of the centrosome by the anti-AZI antibody in NIH/3T3 cells fixed with MeOH; this staining was abolished if the anti-AZI antibody is absorbed with purified GST-AZI, which indicated that the centrosomal signal was specific for AZI. The centrosomal localization was only seen in cells fixed with formaldehyde during mitosis, but not during other phases of the cell cycle. However, when cells were fixed with MeOH, centrosomal localization was seen in every cell and in every phase of the cell cycle. Thus, the centrosomal localization of AZI is best visualized using this mouse monoclonal antibody when the cells are fixed with methanol.

We also showed the centrosomal and nuclear localization of AZI in MeOH-fixed human endothelial (HUVEC) and osteosarcoma cells (U-2OS).

The localization of AZI to active centrosomal fractions of U2-OS cells was analyzed using centrosomes prepared from sucrose gradient fractions (Mitchison and Kirschner, Meth. Enz. 134:261-9 (1986)). Sequential gradient fractions were run on SDS-PAGE gels and the expression of different proteins was analyzed by Western blot. The above-described mouse monoclonal antibody was used to detect AZI; Centrin-GFP (stably expressed in this cell line) was detected using an anti-GFP antibody; and the centrosome-specific tubulin, gamma-tubulin, was detected using an anti-gamma tubulin antibody. The centrosomes were found in fraction 4 of this gradient, as indicated by the expression of both tubulin and the centrosomal protein Centrin. AZI was also found in fraction 4, confirming its localization to the centrosome.

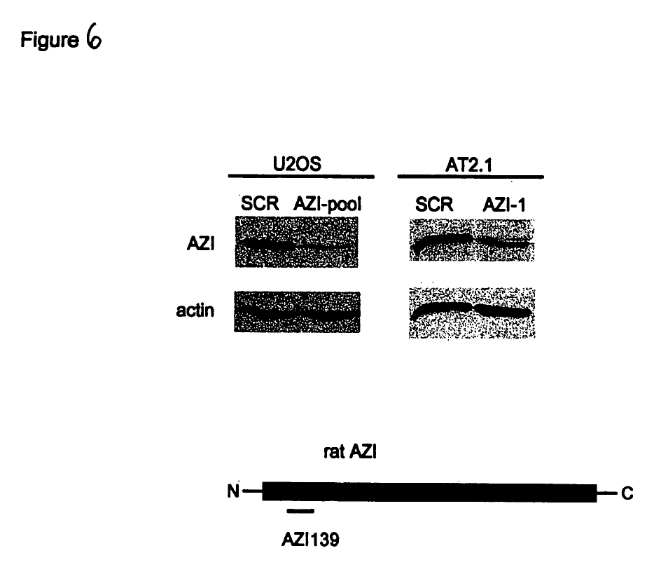

FIG. 6 shows that silencing of AZI leads to a decrease in centrosome abnormalities. U2OS and AT2.1 cells were transiently transfected with siRNA against AZI (AZI-139 and AZI-pool) and harvested after 48 h. AZI139 specifically targets an N-terminal sequence of the rat AZI coding sequence. The commercially available AZIpool (Smartpool) consists out of four pooled siRNAs directed against human AZI. Immunoblot analysis shows that AZI protein expression is greatly reduced in siRNA-treated cells. A non-specific scrambled siRNA was used as control (SCR).

To further characterize the effect of RNAi-AZI on NIH/3T3 cells, we showed that such cells have micronuclei.

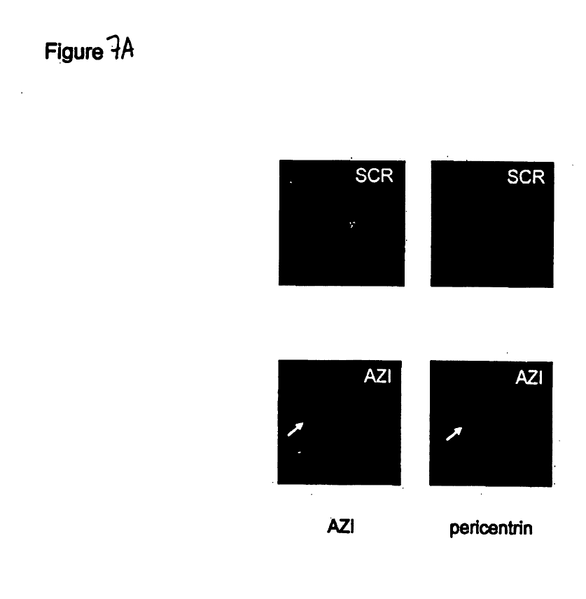

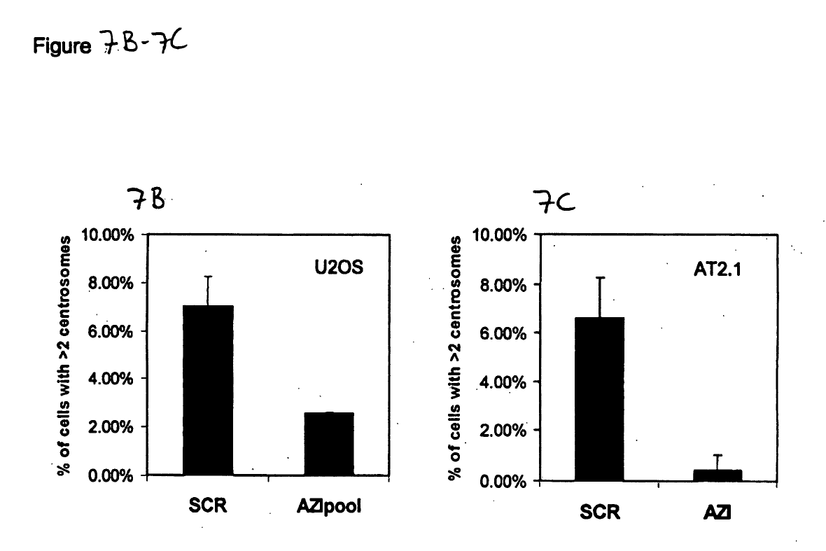

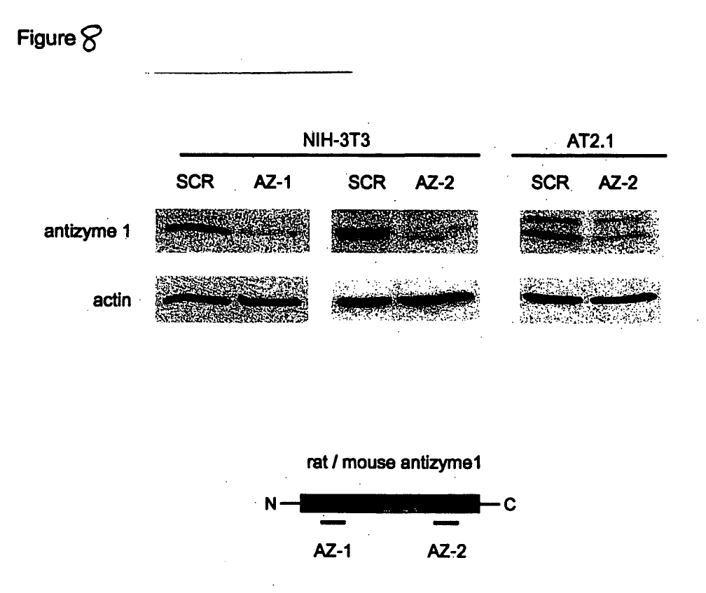

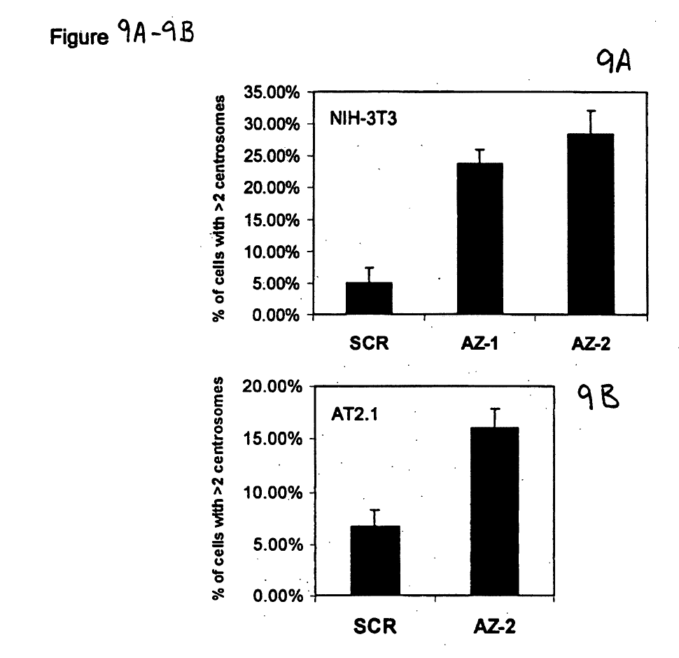

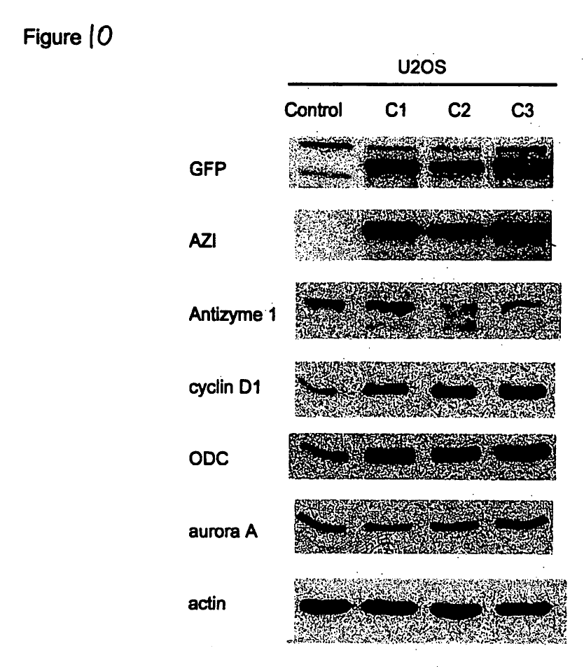

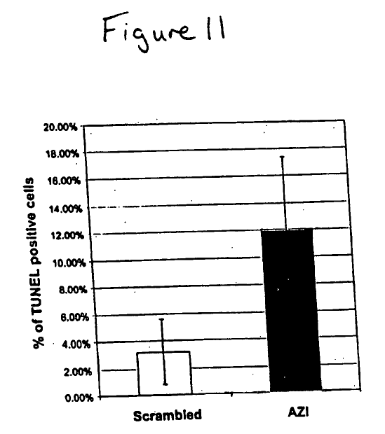

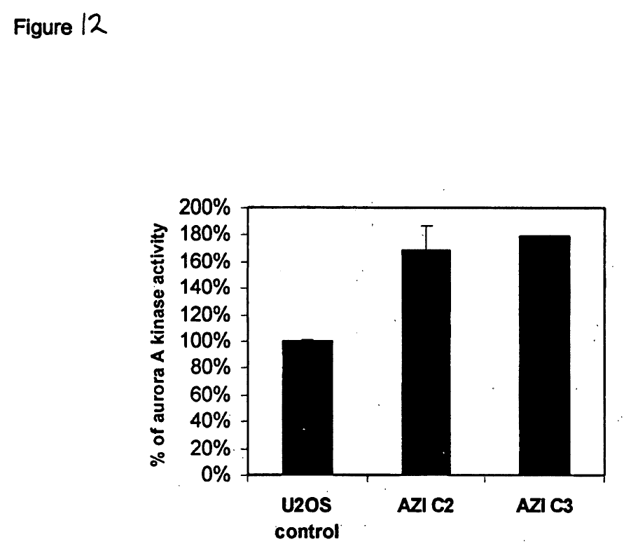

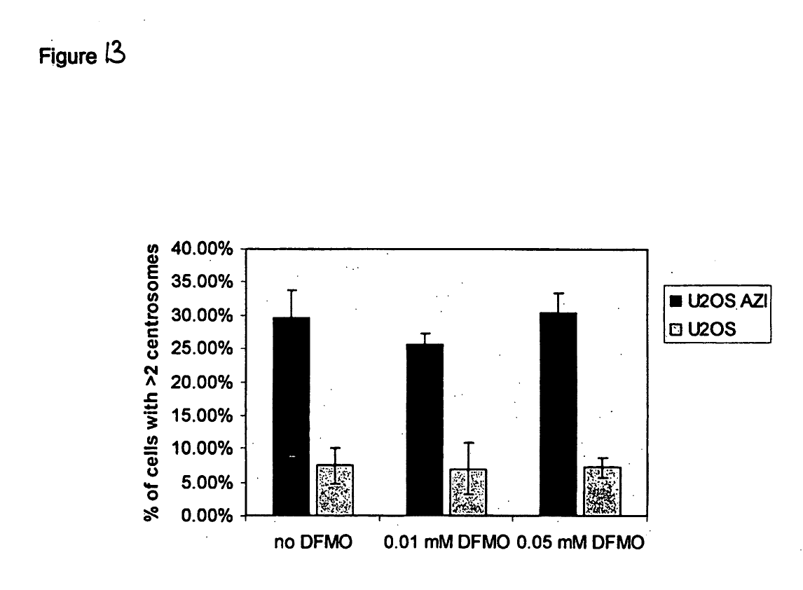

We showed that cells transfected with RNAi-AZI have an increase in the number of abnormal centrosomes. FIG. 7A shows immunofluorescence analysis of AZI levels in U2OS cells treated with siRNA against AZI or control siRNA (SCR). Cells were stained for AZI (mouse monoclonal) and pericentrin (rabbit polyclonal). The upper panel shows centrosomal AZI signal in a cell treated with scrambled siRNA. The lower panel shows a cell which was treated with siRNA against AZI and has undetectable AZI expression at the centrosome (arrow). The level of AZI knockdown was similar to the results obtained by immunoblot analysis. FIGS. 7B and 7C show quantitative analysis of centrosome abnormalities in U2OS and AT2.1 cells treated with siRNA against AZI (AZI-pool, AZI139) or a scrambled control siRNA. Cells were plated on coverslips, treated with the corresponding siRNAs the following day and fixed 48 h later. Centrosomes were visualized by immunofluorescence staining for γ-tubulin. Bars represent the mean of two independent experiments±s.d. At least 100-150 cells were evaluated per experiment.

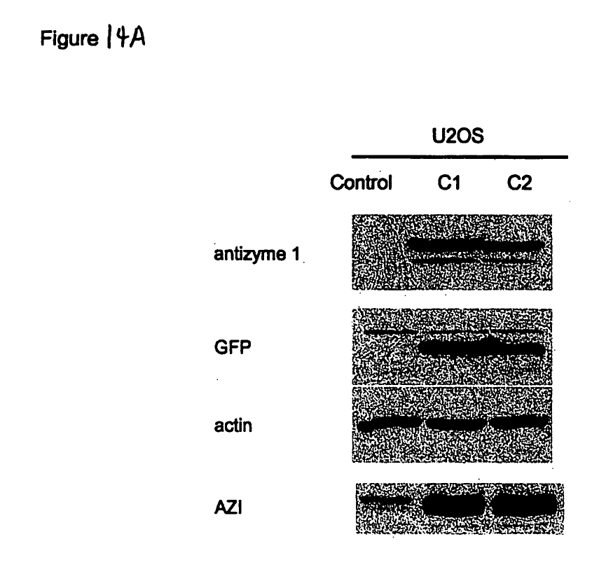

FIG. 8 shows that silencing of antizyme leads centrosome amplification. NIH-3T3 and AT2.1 cells were transiently transfected with siRNA against antizyme 1 (AZ-1 and AZ-2) and harvested 48 h later. AZ-1 specifically targets an N-terminal sequence of the rat/mouse antizyme 1 and AZ-2 targets a C-terminal sequence of the rat/mouse antizyme 1. Immunoblot analysis shows that antizyme 1 protein expression is greatly reduced in siRNA-treated cells. A non-specific scrambled siRNA was used as control (SCR).

We showed that human foreskin fibroblasts transfected with AZI-RNAi have an increase in the number of cells with abnormal centrosome numbers. Cells were transfected with RNAi against AZI or scrambled RNAi, put into a 60 mm dish and transferred the next day into a 24-well plate. The remaining cells were placed into two 35 mm dishes, for Western blotting. Immunostaining was performed two days after transfection. FIG. 9 shows quantitation of this data.

We also showed centrosome and spindle abnormalities in human foreskin fibroblasts transfected with AZI-RNAi using immunohistochemistry with anti-gamma-tubulin antibody GTU-88. We saw cells with an abnormal centrosome numbers and cells with abnormal spindles.

We showed that silencing AZI leads to growth inhibition in AT2.1 cells.

FIG. 10 shows stable overexpression of AZI leads to centrosome amplification in U2OS cells. Immunoblot analysis of GFP-AZI overexpressing U2OS cells. Three independent GFP-AZI clones (C1-3) were analysed using antibodies against GFP, AZI (mouse monoclonal), cyclin D1, ODC, antizyme 1 and aurora A. Actin was used as loading control. The negative control represents lysate from parental U2OS cells.

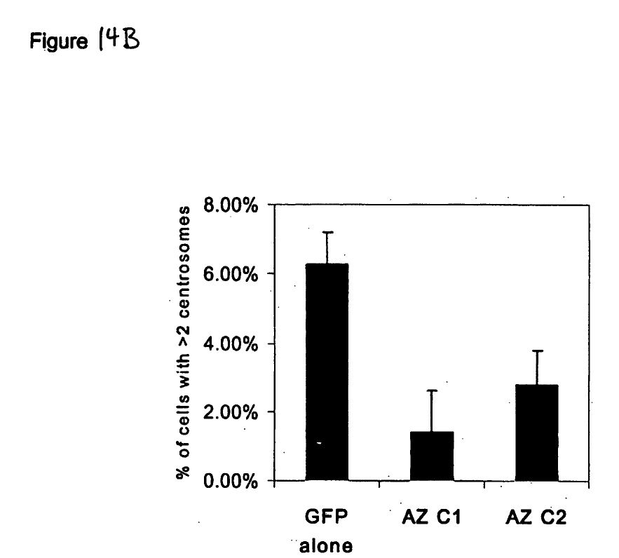

We also showed increased apoptosis in NIH/3T3 cells treated with AZI-RNAi. FIG. 11 shows a TUNEL assay of cells treated for 48 hours with AZI-RNAi or a scrambled control RNA. FIG. 30B shows TUNEL staining in cells treated with AZI-RNAi (left hand) or a scrambled control RNAi (right hand).

We showed that short term overexpression of AZI prevents centrosome duplication. In this experiment, NIH/3T3 cells were transfected with pcDNA3.1 or pcDNA3.1-AZI (Myc-tagged human AZI cloned into pcDNA3.1). Cells were fixed with MeOH 24 hours after transfection, and the percentage of cells with 1 or 2 centrosomes was determined.

RNAi (SMARTpool, Dharmacon) against AZI or scrambled RNAi was used on primary human foreskin fibroblasts. 0.5×10⁶ cells were transfected with RNAi (scrambled or AZI) and directly plated into a 60 mm dish. Phase pictures were taken after 48 h before cells were harvested for western blot. A Western blot of the same primary human foreskin fibroblasts treated with RNAi against AZI was also performed.

These results show that silencing of AZI leads to growth inhibition in primary cells (human foreskin fibroblasts) indicating that AZI is essential for proliferation. The Western blot confirms that RNAi-AZI treated cells have a lower level of AZI than cells treated with the control scrambled RNAi.

FIG. 12 shows parental U2OS cells and two AZI-overexpressing clones were analysed for aurora A kinase activity. Lysates of non-synchronized cells were subjected to a non-radioactive immunoassay for aurora A kinase activity using an anti-phospho-Lats2 serine83 monoclonal antibody and peroxidase-coupled anti-mouse antibody as a reporter molecule. Assay was repeated with all samples assayed in duplicates.

FIG. 13 shows AZI overexpressing cells (clone 3) and parental U2OS cells were grown in 0.5% FBS containing media and were treated for 24 hours with either 0.01 mM or 0.05 mM DFMO Polyamine levels were measured as described in Materials and Methods. Mean values from duplicate samples±s.d. are shown.

FIGS. 14A and 14B show that overexpression of antizyme 1 leads to a decrease in centrosome abnormalities. FIG. 14A shows that immunoblot analysis of GFP-antizyme 1 overexpressing U2OS cells. Two independent GFP-antizyme 1 clones were analysed using antibodies against GFP or antizyme 1 (mouse monoclonal). Actin was used as loading control. The negative control represents lysate from parental U2OS cells. FIG. 14B shows quantitative analysis of centrosomal abnormalities in U2OS cells stably overexpressing GFP-antizyme 1. Two independent antizyme 1 overexpressing clones were analysed for centrosome abnormalities. The combined result of three independent stable GFP clones was used as control. Centrosomes were stained for γ-tubulin and visualized by immunofluorescence. Bars represent the means of three independent experiments±s.d.

Treatment with hydroxyurea is known to cause centrosome amplification in some cell-lines, including U2OS, generating multiple centrosomes per cell (Nigg, 2002, Nat Rev Cancer). The mechanism for this centrosome hyperamplification is unknown, however. We therefore examined whether either antizyme 1 overexpression or reduced AZI expression could affect the response of U2OS cells to hydroxyurea (HU). Since we had seen AZI stabilization in the antizyme 1 overexpressing clones we also combined both antizyme overexpression and AZI knockdown. U2OS wild-type and antizyme overexpressing cells were transfected with siRNA against AZI or non-specific scrambled siRNA as a control. These cells were then treated with 2 mM hydroxyurea for 48 h. As seen, treatment with siRNA against AZI reduced centrosome hyperamplification in wild-type U2OS cells. Also antizyme 1 overexpression results in a lower percentage of centrosome abnormalities compared to wild-type U2OS cells. If antizyme 1 overexpression and silencing of AZI are combined, U2OS cells show a strong reduction in centrosome abnormalities in the presence of hydroxyurea. Taken together these results suggest that the antizyme 1/AZI pathway has an important function in the regulation of centrosome numbers with the presence of antizyme 1 correlating with centrosome normalization and upregulation of AZI correlating with centrosome amplification. FIG. 15 shows quantitative analysis of centrosome abnormalities in U2OS cells upon treatment with hydroxyurea. U2OS wild-type and U2OS-antizyme (U2OS-AZ) overexpressing cells were transfected with siRNA against AZI (Smartpool) or scrambled control siRNA. 24 h after transfection cells were treated with 2 mM hydroxyurea for an additional 48 h. Centrosomes were visualized by immunofluorescence staining for γ-tubulin. Bars represent the mean of two independent experiments±s.d. At least 150-200 cells were evaluated for each experiment.

We showed that overexpression of antizyme 1 leads to a decrease in centrosome abnormalities. We performed an immunoblot analysis of GFP-antizyme 1 overexpressing U2OS cells. Two independent GFP-antizyme 1 clones were analysed using antibodies against GFP or antizyme 1 (mouse monoclonal). Actin was used as loading control. The negative control represented lysate from parental U2OS cells.

We also performed a quantitative analysis of centrosomal abnormalities in U2OS cells stably overexpressing GFP-antizyme 1. Two independent antizyme 1 overexpressing clones were analysed for centrosome abnormalities. The combined result of three independent stable GFP clones was used as control. Centrosomes were stained for γ-tubulin and visualized by immunofluorescence. Bars represent the means of three independent experiments±s.d.

AZI Localizes to Centrosomes in Mouse, Rat and Human Cells

Several components of the ubiquitin-dependent degradation pathway localize to the centrosome (Doxsey et al., 2005). Therefore, we were interested to see if also proteins such as antizyme 1 and AZI which are involved in ubiquitin-independent degradation may show centrosomal localization. We first performed immunofluorescence microscopy for AZI in various mouse, rat and human cell lines as well as in primary human cells using a mouse monoclonal antibody against AZI (Murakami et al., 1989). Immunostaining for AZI was detectable as cytoplasmic, membrane associated or nuclear depending on the cell type and method of fixation (data not shown). However, in mitotic cells we consistently detected AZI staining at the spindle poles independent of the method of fixation. Moreover, using methanol fixation we consistently detected one or two bright spots in non-mitotic cells that were close to the nuclear envelope suggesting that AZI might be localized at the centrosome. We therefore performed colocalization experiments using an antibody against the known centrosomal marker pericentrin (Doxsey et al., 1994). As shown by immunofluorescence microscopy, AZI and pericentrin colocalize in a variety of mammalian cell lines (FIG. 1a) including human osteosarcoma cells (U2OS), which is a cell line commonly used for centrosome studies, mouse fibroblasts (NIH-3T3) and Dunning rat prostate carcinoma cells (AT2.1). AZI also localizes to centrosomes in primary human foreskin fibroblasts (HFF) and human umbilical vein endothelial cells (HUVEC). In addition to its centrosomal localization, AZI was also present in the cytoplasm, nucleus and in Golgi-like structures depending on the cell type analysed. Centrosomal AZI localization was also supported by confocal microscopy showing colocalization of AZI with pericentrin (data supplement). In addition, the specificity of the centrosomal AZI signal was confirmed with a second AZI antibody using a rabbit polyclonal antibody raised against a C-terminal AZI peptide. We detected specific colocalization of the AZI signal with the centrosomal marker protein γ-tubulin.

To further confirm the specificity of the AZI signal, we performed competition studies. Purified GST or GST-tagged rat AZI protein was added together with the primary antibody during immunostaining. Addition of GST-tagged AZI completely abolished the centrosomal AZI staining whereas addition of GST alone did not change the AZI staining pattern. The remaining weak cytoplasmic signal is due to non-specific components of the primary antibody and was not seen with the secondary antibody alone. In addition, the centrosomal localization of AZI was also confirmed by immunoblot analysis of centrosomal fractions derived from U2OS cells stably overexpressing a GFP-tagged version of the centriole marker centrin (Piel et al., 2000). As seen in FIG. 1d, AZI cofractionated with the centrosomal marker proteins γ-tubulin and centrin.

We next analysed the centrosomal localization of AZI in NIH-3T3 cells throughout the cell cycle. As shown in FIG. 1e, AZI localizes to centrosomes at each phase of the cell cycle. During interphase, a specific AZI signal was detected for both of the two centrosomes. This staining pattern was very similar to that of γ-tubulin. By metaphase, both centrosomes had high levels of AZI and were more brightly stained than at any other cell cycle stage. At the metaphase to anaphase transition, AZI staining diminished and reached its lowest levels by late anaphase/telophase. During telophase, an AZI signal also appeared adjacent to the intercellular bridge where the maternal centriole repositions during cytokinesis (Piel et al., 2001). Treatment with the microtubule-depolymerising drug nocodazole did not displace the centrosomal AZI signal demonstrating that the localization of AZI to the centrosome is independent of microtubule dynamics (data not shown).

Centrosomal Localization of Antizyme 1

We next wanted to address the question whether the known AZI binding partner antizyme 1, localizes to centrosomes as well. We performed immunostaining using the □ tubulin antibody and a polyclonal rabbit antibody against antizyme 1 (Mitchell et al., 1996). As seen in FIG. 2a, we detected colocalization of □-tubulin with the antizyme 1 signal in mouse, rat and human cells demonstrating that also antizyme 1 colocalizes to centrosomes. In contrast to AZI, the centrosomal signal for antizyme 1 was strongest during interphase and was mostly absent during mitosis. In addition to its centrosomal localization, antizyme 1 was also present in the nucleus, as has been reported previously (Schipper et al., 2004; Murai et al., 2003). Within the nucleus, antizyme 1 was concentrated in vesicle like structures but excluded from nucleoli. Some cells, however, showed only a weak nuclear antizyme 1 signal. To further confirm the specificity of the centrosomal antizyme 1 signal we cloned rat antizyme 1 into the pEGFP C3 expression vector and established U2OS cells stably overexpressing GFP-tagged antizyme 1. Immunostaining for GFP and γ-tubulin confirmed the centrosomal localization of GFP-tagged antizyme 1. Unequivocal centrosomal localization of GFP-antizyme 1 was seen at low expression levels. In cells expressing higher levels, GFP-antizyme 1 also localized to the nucleus and additional diffuse staining was seen throughout the cytoplasm. The centrosomal localization of antizyme 1 was also confirmed by confocal microscopy, demonstrating colocalization of endogenous antizyme 1 or GFP-tagged antizyme 1 with γ-tubulin (data supplement). In addition, the centrosomal localization of antizyme 1 was detected by immunoblot analysis of centrosomal fractions derived from U2OS cells. Antizyme 1 cofractionated with the centrosomal marker protein γ-tubulin. The existence of two antizyme 1 bands has been reported previously and represents initiation at the first and second AUG codon of the open reading frame (ORF) 1 of antizyme 1 (Matsufuji et al., 1995; Mitchell et al., 1998). Taken together, these data strongly suggest that antizyme 1 localizes to centrosomes in mammalian cells.

Preferential Localization of Antizyme 1 to the Maternal Centriole

Strikingly, a preferential localization of antizyme 1 with one of the two centrosomes was repeatedly observed in several mammalian cell lines. During mitosis, cells inherit a single centrosome that contains a pair of centrioles. The older “mother” centriole, which was formed at least one and a half generations earlier, is slightly larger and has appendages whereas the younger “daughter” centriole, formed during the previous S-phase, lacks these structures (Piel et al., 2000). In addition, the maternal centriole remains near the cell center while the daughter migrates extensively throughout the cytoplasm (Piel et al., 2000). We observed that antizyme 1 is associated with the brighter, less mobile centrosome and showed a staining pattern similar to ninein, which is a known marker of the maternal centriole (data not shown) (Mogensen et al., 2000). Another marker for the maternal centriole is acetylated □-tubulin (Lange et al., 1995). Centriole distribution is semi-conservative and the primary cilium, which contains acetylated □-tubulin remains with the maternal centriole and is resistant to depolymerisation by nocodazole. In addition, the primary cilium can be made visible in response to serum starvation in NIH-3T3 cells. Hence, we tested if antizyme colocalizes with the primary cilium. NIH-3T3 cells were treated with serum starvation or with nocodazole and then co-stained for acetylated □-tubulin and antizyme. As seen in FIG. 2 d antizyme 1 staining colocalizes with the primary cilium and is absent from the daughter centrosome in serum-starved NIH-3T3 cells. This strongly suggests that antizyme 1 preferentially associates with the maternal centrosome. In addition, treatment with the microtubule-depolymerising drug nocodazole did not displace the centrosomal antizyme 1 signal demonstrating that the centrosomal localization of antizyme 1 is microtubule independent.

Silencing of AZI or Antizyme 1 Modulates Centrosome Amplification

To further evaluate the centrosomal function of AZI, we reduced its protein expression level using siRNA. AZI was silenced by two different sets of siRNA (AZI-139 and AZI-pool). AZI139 specifically targets an N-terminal sequence of the rat AZI coding sequence. The commercially available AZIpool (Smartpool) consists of four pooled siRNAs directed against human AZI. A non-specific scrambled siRNA was used as control (SCR). Rat prostate carcinoma cells (AT2.1) and human osteosarcoma cells (U2OS) were transiently transfected with the rat and human AZI-specific siRNAs respectively, and harvested after 48 h. As confirmed by immunoblot analysis, each of the siRNAs caused a marked reduction in AZI protein levels in the respective cell type. AZI protein levels were reduced 77 88% as quantified by imaging software.

We next examined whether AZI silencing affected centrosome amplification in these tumour cell lines. We plated U2OS and AT2.1 cells onto coverslips and treated the cells for 48 h with siRNA against AZI or with the control siRNA. Immunofluorescence microscopy demonstrated that the centrosomal staining for AZI was strongly reduced in cells transfected with siRNA against AZI. Centrosomes were then stained with anti γ-tubulin antibody and centrosome numbers were quantified. Cells with more than two centrosomes were considered abnormal. Silencing AZI in these tumour cell lines rapidly led to a significant decrease (P=0.00221, U2OS; P=0.000289, AT2.1) in cells with abnormal centrosome numbers indicating that AZI silencing might normalize centrosome copy levels in tumour cells.

We next wanted to determine whether loss of antizyme 1, which is the major AZI binding partner and a reported tumour suppressor, would also influence centrosome amplification. We silenced antizyme 1 in NIH-3T3 mouse fibroblasts and in AT2.1 cells using two different siRNAs directed against separate sequences of antizyme 1 called AZ-1 and AZ 2. Both siRNAs caused a strong downregulation (75 90% reduction) of antizyme 1 protein expression as quantified by imaging software. Reduced antizyme 1 levels correlated with an increase in cells with abnormal centrosome numbers (P=0.00193, NIH-3T3; P=0.001126, AT2.1). Together these results suggest an important role for antizyme 1 and AZI in modulating centrosome numbers in a variety of cell lines.

Stable Ectopic Expression of AZI

High level expression of AZI was observed previously in a variety of tumour cell lines (Jung et al., 2000) suggesting a possible correlation between AZI and malignancy. To investigate whether ectopic overexpression of AZI does effect centrosome amplification, we established U2OS clones that stably express GFP-AZI. The N-terminal GFP-tag did not interfere with the ability of AZI to bind antizyme 1. The overexpression of GFP-tagged AZI was confirmed by immunoblotting using antibodies against AZI or GFP, respectively. We could also detect centrosomal localization of GFP-AZI by immunofluorescence.

Interestingly, all three AZI overexpressing clones showed a strong increase in cyclin D1 levels and in the well-known antizyme target protein ODC. As we recently reported that antizyme 1 can promote the degradation of cyclin D1 (Newman et al., 2004), the effect on cyclin D1 may be due to antizyme 1 inactivation in the AZI overexpressing cells. Since there are, however, conflicting reports on the connection between cyclin D1 and centrosome function (Nelsen et al., 2005; Spruck et al., 1999) we wished to assess whether an important centrosome-associated kinase such as aurora A might be affected by AZI overexpression as well. Total aurora A protein levels remained unchanged in AZI overexpressing cells as shown by immunoblot analysis (FIG. 10). We then tested for modulation of aurora A kinase activity in the AZI stable clones. Aurora A kinase activity is significantly increased in the AZI overexpressing cells compared to wild type U2OS cells (P=0.00126). Thus AZI overexpression is correlated with increases in both cyclin D1 and ODC levels as well as aurora A kinase activity potentially leading to hyperproliferation and centrosomal amplification.

AZI Overexpression Results in Centrosome Amplification

We next examined the effect of AZI overexpression on centrosome numbers. Presumably, overexpression of AZI should have a similar effect on centrosome amplification as loss of antizyme 1 and should lead to an increase in centrosome abnormalities. Cells were stained for γ-tubulin and centrosome numbers were quantified. As a control, we established U2OS cells that stably overexpressed GFP alone and quantified the centrosome abnormalities of three independent GFP clones. All three clones analysed showed the same percentage of centrosome abnormalities as wild type U2OS cells. In contrast, AZI overexpressing cells showed a significant increase in centrosome abnormalities (P=0.0000712). AZI overexpressing cells showed a 3.5-5 fold increase in centrosome abnormalities when compared to control cells that stably overexpressed GFP alone.

Since AZI-overexpressing cells demonstrated a strong increase in ODC protein we wanted to analyze the polyamine levels in the AZI overexpressing cells. ODC is the key enzyme in polyamine biosynthesis and catalyses the decarboxylation of ornithine to putrescine. As expected, a strong increase in putrescine levels was observed in the AZI overexpressing cells whereas spermidin and spermine levels remained more or less stable.

We therefore wanted to test the hypothesis that high putrescine levels might contribute to the centrosome amplification observed in the AZI overexpressing cells. We therefore treated parental and AZI overexpressing cells with the known ODC inhibitor DFMO and analysed centrosome abnormalities in these cells. Reduction in ODC activity by DFMO does not lead to a decrease in centrosome abnormalies in neither the parental nor the AZI overexpressing cells, suggesting that high putrescine levels are not responsible for the observed centrosome abnormalities of AZI overexpressing cells.

In addition we also tested the hypothesis that the observed centrosome abnormalities in the AZI overexpressing cells are a result of cytokinesis failure and assessed the ploidy status of the AZI overexpressing cells by flow cytometry. The DNA-content of AZI overexpressing cells was identical to that of wild-type U2OS cells suggesting that AZI overexpression does not lead to defects in cytokinesis.

Stable Ectopic Expression of Antizyme 1 and its Influence on Centrosome Amplification

We next investigated the consequence of antizyme 1 overexpression. If the effects of AZI and antizyme 1 are coupled, then overexpression of antizyme 1 would have an effect on centrosome amplification comparable to loss of AZI and should lead to a decrease in centrosome abnormalities. As reported before we generated U2OS clones that stably overexpressed GFP-antizyme 1, with the GFP-tag located at the N-terminus of antizyme 1. Again, the N-terminal GFP-tag did not interfere with the ability of antizyme 1 to bind AZI (data not shown). The expression of GFP-antizyme 1 in two stable U2OS clones was verified by immunoblotting. Interestingly, we observed also a strong induction of AZI in the antizyme overexpressing clones. Analysis of polyamine levels in the antizyme overexpressing clones showed only a modest reduction of putrescine compared to the parental cells. This indicates that there is a strong compensation of antizyme by the induction of AZI. In general antizyme 1 overexpressing clones grew at a lower proliferative rate than the parental U2OS cells. This was expected since overexpression of antizyme 1 has been shown to inhibit cell proliferation. We then analysed two antizyme 1 overexpressing clones for centrosome abnormalities. Again cells were stained for γ-tubulin and scored for centrosome numbers. Antizyme 1 overexpressing cells showed a 2-4.5 fold decrease in centrosome abnormalities when compared to controls that where stably overexpressing GFP alone. Taken together these results suggest that the antizyme 1/AZI pathway acts to regulate centrosome numbers with the presence of antizyme 1 correlating with centrosome normalization and the upregulation of AZI correlating with centrosome amplification.

We have identified in this study that antizyme 1 and AZI are core components of the centrosome and that they function to regulate centrosome amplification. AZI and antizyme 1 localize to centrosomes in a variety of cell lines and primary cells. Both proteins do not exclusively localize to centrosomes but a fraction of endogenous AZI and antizyme 1 localized to centrosomes and mitotic spindle poles in every cell type analysed. Immunofluorescent analyses with the centrosomal marker proteins γ-tubulin and pericentrin and analysis of purified centrosome fractions confirmed that AZI and antizyme 1 are true components of the centrosome. In addition, centrosomal localization of AZI and antizyme 1 was also observed with the GFP-tagged version of both proteins. In the case of AZI, additional control experiments involving a second antibody against AZI or competition studies further support that AZI is a centrosomal protein.

GFP-tagged antizyme 1 colocalized with centrosomes in stably transfected U2OS cells. The centrosomal antizyme signal, however, had to be amplified with a GFP antibody suggesting that antizyme 1 is not an abundant centrosomal component. Antizyme 1 promotes the degradation of ODC through the 26S proteasome in an ubiquitin-independent manner. During this process antizyme 1 is released and can participate in subsequent cycles of protein degradation. Therefore, even a low abundance of antizyme 1 at the centrosome could promote multiple rounds of antizyme 1 target protein degradation. In this context it is interesting to note that several components of the ubiquitin-dependent degradation pathway localize to the centrosome (Doxsey et al., 2005) and that purified centrosome preparations contain active 26S proteasomes (Fabunmi et al., 2000). These data provide evidence for the importance of proteasomal degradation at this organelle. Here we present evidence that centrosomes also contain elements of the ubiquitin-independent, antizyme-mediated protein degradation pathway as well.

Another important function of antizyme 1 might involve centrosome nuclear shuttling (Doxsey et al., 2005). There are only a few proteins known so far including centrin or RAD51 that show dual localization to the nucleus and the centrosome (Daboussi et al., 2005; Araki et al., 2001). Cell cycle and centrosome duplication are synchronized events and centrosome nuclear shuttling could be used to create a signal at one organelle that is required for the function of the other. Alternatively, events could be synchronized by placing components of both pathways to the nucleus and the centrosome respectively (Doxsey et al., 2005). Antizyme 1 might be one such protein since it localizes to both compartments and does regulate the stability of cell cycle proteins such as cyclin D1.

Our results show that antizyme 1 is preferentially associated with one of the two centrosomes. Since the known antizyme binding partner AZI localizes equally to both centrosomes this distribution pattern might be caused by yet unknown antizyme 1 binding partners and not by the AZI-antizyme interaction. The preferred localization of antizyme 1 with the maternal centrosome also suggests that antizyme 1 may be important for the stability of maternal centrosome components during centrosome duplication and maturation. We have also consistently observed that the centrosomal antizyme 1 signal is most prominent during interphase and decreases as cells approach mitosis, which is in agreement with the observation that antizyme 1 is particularly active during the G1-S-phase of the cell cycle (Oredsson, 2003) during which centrosome amplification and maturation occurs.

It has not been shown yet whether the best studied antizyme 1 binding partner, ODC, localizes to centrosomes as well and we are currently investigating this possibility. To date, we have not been able to detect centrosomal localization for ODC, although this could be due to technical issues with the currently existing ODC antibodies.

Antizyme 1 was shown recently to bind and facilitate the proteasomal degradation of cyclin D1 (Newman et al., 2004). Our results reveal that human osteosarcoma cells that stably overexpress AZI have significantly increased levels of cyclin D1 and ODC. We speculate that high levels of AZI may lead to a reduction in antizyme 1 activity resulting in stabilization of the antizyme target proteins cyclin D1 and ODC. Interestingly, it was recently reported that cyclin D1 overexpression induces centrosome amplification and mitotic spindle abnormalities (Nelsen et al., 2005). An earlier report, however, indicated that cyclin D1 overexpression does not lead to any increase in cells with abnormal centrosome numbers (Spruck et al., 1999). Thus the role for cyclin D1 as a mediator of AZI-induced centrosomal abnormalities is not yet certain. Antizyme 1 may, however, also regulate the stability of other centrosomal components which have not been identified previously such as centrosomal kinases or cell cycle regulators. Therefore, we wanted to investigate the effect of AZI on an important centrosomal constituent and examined whether the major centrosome-associated cell cycle kinase, aurora A, is altered in AZI overexpressing cells. Aurora A kinase localizes to centrosomes and spindle poles and is amplified in a variety of malignant tumours (Marumoto et al., 2005; Meraldi et al., 2004). We observed that stable overexpression of AZI led to an increase in aurora A kinase activity. Though we cannot explain yet how AZI regulates aurora A kinase activity it could involve stability of protein complexes, which may contain AZI, antizyme 1 and aurora A. Together our data suggest that an increase in cyclin D1 levels as well as elevated aurora A kinase activity may contribute to the centrosome abnormalities observed in the AZI overexpressing cells.

In summary, without wishing to be bound by a theory, our results demonstrate that AZI and antizyme 1 activities are necessary for correct centrosome amplification. We therefore propose a model whereby antizyme 1 functions to promote the proper degradation of centrosomal components or cell cycle regulators which are responsible for maintaining normal centrosome structure and function. By affecting antizyme 1 activity, upregulated AZI could lead to centrosomal abnormality. We suggest that alterations in intracellular balance of antizyme 1 and AZI contribute to tumour-associated centrosomal amplifications.

We claim:

1. A method for inhibiting antizyme inhibitor (AZI) in a cell, the method comprising contacting a cell with an inhibitor of AZI.

2. The method of claim 1, wherein said inhibitor of AZI inhibits AZI activity and/or expression.

3. The method of claim 2, wherein said inhibitor of AZI comprises an antibody.

4. The method of claim 2, wherein said inhibitor of AZI comprises an RNA interference molecule.
